(19) United States
(12) Patent Application Publication
(10) Pub. No.: US 2007/0015913 A1
(43) Pub. Date: Jan. 18, 2007

(54) Mrp3 genes and uses thereof

(75) Inventors: Lan Bo Chen, Lexington, MA (US); Daniel Auclair, Ashland, MA (US)

Correspondence Address: FISH & RICHARDSON PC, P.O. BOX 1022, MINNEAPOLIS, MN, 55440-1022, US

(73) Assignee: DANA-FARBER CANCER INSTITUTE, INC., Boston, MA (US)

(21) Appl. No.: 10/542,128

(22) PCT Filed: Feb. 24, 2004
(86) PCT No.: PCT/US04/05335

§ 371 Date: Jan. 27, 2006

Related U.S. Application Data
(60) Provisional application No. 60/450,071, filed on Feb. 25, 2003.

(51) Int. Cl. C12Q 1/68; C07H 21/04; C07K 14/82; C07K 16/46; A61K 48/00

(52) U.S. Cl. 536/23.5; 435/6; 435/69.1; 435/320.1; 435/325; 514/44; 530/350; 530/388.22

(57) ABSTRACT

A nucleic acid capable of driving the expression of a reporter gene in a cancer cell line is disclosed. A polypeptide able to confer multidrug-resistance on a cell is disclosed. Also disclosed are nucleic acids encoding the polypeptide, expression vectors, transformant host cell lines expressing the polypeptide, and antibodies binding to the polypeptide. Screening, diagnostic and treatment methods using the above polypeptide, nucleic acids, antibodies and host cell lines are also within the scope of this invention.

### BACKGROUND

Systemic therapy with cytotoxic drugs or chemotherapeutic agents is the basis for many treatments of disseminated cancers. Most cancers are highly responsive to initial treatments but more often become resistant to further therapy. It is common that these cancers develop resistance to more than one particular chemotherapeutic agent with which the cancers are treated, i.e., multidrug-resistance. It is also common that some cancers are intrinsically resistant to most chemotherapeutic agents (e.g., non-small cell lung cancer, malignant melanoma and colon cancer). For these multidrug-resistant cancers, chemotherapy is effective only in a minority of cases. As a result, success with conventional salvage chemotherapies has been limited (Morrow C S et al., Drug Resistance and Its Clinical Circumvention. In: Cancer Medicine, Holland J F, Frei E, Bast R C et al., (eds.) Vol. 1, pp. 799-815, 1997. Williams & Wilkins, Baltimore, Md.).

Studies on the molecular basis of the above-mentioned multidrug-resistance in cultured cell lines have revealed multidrug-resistant cells differ from drug-sensitive cells in a number of ways, including: (a) a reduced accumulation of cytotoxic drugs due to decreased drug influx and/or increased drug efflux; (b) altered drug metabolism; (c) increased DNA repair; (d) altered drug targets; and (e) altered expression and/or activity of certain cellular proteins. The most commonly reported alteration in multidrug-resistant cancer cells has been the increased expression of the 170 kDa plasma membrane glycoprotein, P-glycoprotein (Pgp), which is encoded by the multidrug-resistance 1 (MDR1) gene. Pgp is a member of a superfamily of membrane proteins that serve to transport a variety of molecules, ranging from ions to proteins, across cell membranes. This superfamily is known as the ATP-binding cassette (ABC) superfamily of membrane transport proteins. For a review see Higgins C F, Ann Rev Cell Biol 8:67, 1992.

Studies on clinical samples and cell lines representing many cancer types have shown that Pgp, while clinically relevant in some malignancies, is unlikely to be important in others. For example, overexpression of Pgp is an infrequent occurrence in small cell lung cancer and non-small cell lung cancer, both of which are multidrug-resistant. The multidrug-resistance mechanisms identified so far in vitro can explain only a small proportion of clinical multidrug-resistance.

Further, unlike clinical multidrug-resistance, the in vitro multidrug-resistance usually does not include resistance to DNA-damaging agents such as platinum-containing compounds, alkylating agents, or antimetabolites (Pastan, I et al., Annu Rev Med 42:277-286, 1991). These DNA-damaging agents represent more than 80% of the drugs used in cancer therapy.

The complexity of the multidrug-resistance is daunting. Few genes that can actually confer in vivo resistance to cancer chemotherapeutic agents have thus far been identified. Thus, the identification of a comprehensive set of multidrug-resistance genes is needed. Further, therapeutics and diagnostics based on such genes would provide additional tools to treat and diagnose cancers associated with these genes.

### SUMMARY

The present invention is based, at least in part, on the discovery of two nucleic acids from multidrug-resistant cancer cell lines (SEQ ID NO:1 and SEQ ID NO:3). The nucleic acid with the sequence of SEQ ID NO:1 encodes a polypeptide designated as Multiple Resistance Protein 3 splice variant 1 (MRP3s1, SEQ ID NO:2). This polypeptide is overexpressed in certain multidrug-resistant cancer cell lines and, when expressed in a drug sensitive mammalian cell, can confer multidrug-resistance on the cell. The nucleic acid with the sequence of SEQ ID NO:3 can drive the expression of a gene in a multidrug-resistant cell. These nucleic acids and the polypeptide represent molecules that can be targeted diagnostically or therapeutically in multidrug-resistant cancers expressing the nucleic acid and polypeptide.

In one aspect, the invention features an isolated nucleic acid that contains a nucleotide sequence at least 70% identical to the sequence of SEQ ID NO:3, as well as its fragments thereof. In another aspect, the invention features an isolated nucleic acid that contains the sequence of SEQ ID NO:3, as well as its fragments thereof. In some embodiments, the isolated nucleic acid can drive the expression of a reporter gene operably linked to it. In some embodiments, the isolated nucleic acid drives the expression of the reporter gene specifically in a drug-resistant cancer cell.

The term “nucleic acid” is intended to include DNA and RNA and can be either double stranded or single stranded. An “isolated nucleic acid” is a nucleic acid the structure of which is not identical to that of any naturally occurring nucleic acid. The term therefore covers, for example, (a) a DNA that has the sequence of part of a naturally occurring genomic DNA molecule but is not flanked by both of the coding sequences that flank that part of the molecule in the genome of the organism in which it naturally occurs; (b) a nucleic acid incorporated into a vector or into the genomic DNA of a prokaryote or eukaryote in a manner such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene, i.e., a gene encoding a fusion protein. Specifically excluded from this definition are nucleic acids present in mixtures of different (i) DNA molecules, (ii) transfected cells, or (iii) cell clones: e.g., as these occur in a DNA library such as a cDNA or genomic DNA library. “Operably linked” or “Operatively linked” means that a nucleic acid is linked to a regulatory sequence in a manner that allows the expression of a gene flanked by the nucleic acid, or of a gene encoded by the nucleic acid. Examples of regulatory sequences include promoters, enhancers and other expression control elements that are known to those skilled in the art. The “reporter gene” means a gene, the product of which can be detected using methods well known in the arts. Examples of such a gene include one encoding a green fluorescent protein, a luciferase, or a lacZ. The “percent identity” of two nucleic acids or of two amino acid sequences is determined using the algorithm of Karlin and Altschul (Proc. Natl. Acad. Sci. USA 87:2264-2268, 1990), modified as in Karlin and Altschul (Proc. Natl. Acad. Sci. USA 90:5873-5877, 1993). Such an algorithm is incorporated into the NBLAST and XBLAST programs of Altschul et al. J. Mol. Biol. 215:403-410, 1990). BLAST nucleotide searches are performed with the NBLAST program, score=100, wordlength=12. BLAST protein searches are performed with the XBLAST program, score=50, wordlength=3. Where gaps exist between two sequences, Gapped BLAST is utilized as described in Altschul et al., Nucleic Acids Res. 25:3389-3402, 1997. When utilizing BLAST and Gapped BLAST programs, the default parameters of the respective programs (e.g., XBLAST and NBLAST) are used. See www.ncbi.nlm.nih.gov.

In some embodiments, the isolated nucleic acid contains a nucleotide sequence encoding a polypeptide with the amino acid sequence of SEQ ID NO:2. In some embodiments, the expression of this polypeptide in a drug sensitive cell renders the cell resistant to cytotoxic drugs, such as a DNA-damaging agent. In some other embodiments, the isolated nucleic acid contains a nucleic acid with the sequence of SEQ ID NO:1 or fragments thereof. A DNA-damaging agent can modify DNA in a way that will affect its reliable replication during cell division. Examples of a DNA-damaging agent include: 1) anthracyclines and other DNA intercalators, e.g., actinomycin D, daunorubicin, doxorubicin, epirubicin, idarubicin, dactinomycin, mitoxantrone, and amsacrine, which possess planar chemical structures and can insert themselves in the space between the successive DNA base pairs, 2) ionizing (such as X-rays and gamma radiation) and ultraviolet radiation that will break chemical bonds in DNA, and 3) alkylating agents and platinum compounds, which can form strong chemical bonds with electron-rich atoms (nucleophiles) such as nitrogen in DNA. Examples of alkylating agents include nitrogen mustards (such as mechlorethamine, melphalan, chlorambucil, cyclophosphamide, and ifosfamide), aziridines, and epoxides (such as thiotepa, mitomycin D, and diaziquone), alkyl sulfonates (such as busulfan and hepsulfam), nitrosoureas (such as carmustine, lomustine, and semustine), and triazenes, hydrazines, and related compounds (such as procarbazine, dacarbazine, and hexamethylamine). Examples of platinum compounds include cisplatin, carboplatin, iproplatin, tetraplatin, and satraplatin), oxaliplatin, and related compounds.

In another aspect, the invention features an isolated nucleic acid having a sequence that, under low, medium, or high stringency conditions, hybridizes to a hybridization probe with the sequence of SEQ ID NO:1 or its complement.

The hybridization technique is well known to one skilled in the art as an alternative method for isolating a nucleic acid encoding a functionally equivalent polypeptide. As used herein, the terms “low stringency,” “medium stringency,”. “high stringency,” or “very high stringency” describe conditions for hybridization and washing. Guidance for performing hybridization reactions can be found in, e.g., Current Protocols in Molecular Biology, John Wiley & Sons, N.Y. (1989), 6.3.1-6.3.6. Aqueous and nonaqueous methods are described in that reference and either can be used. Specific hybridization conditions referred to herein are as follows: (1) low stringency hybridization conditions in 6× sodium chloride/sodium citrate (SSC) at about 45° C., followed by two washes in 0.2×SSC, 0.1% SDS at least at 50° C. (the temperature of the washes can be increased to 55° C. for low stringency conditions); (2) medium stringency hybridization conditions in 6×SSC at about 45° C., followed by one or more washes in 0.2×SSC, 0.1% SDS at 60° C.; (3) high stringency hybridization conditions in 6×SSC at about 45° C., followed by one or more washes in 0.2×SSC, 0.1% SDS at 65° C.; and preferably (4) very high stringency hybridization conditions are 0.5M sodium phosphate, 7% SDS at 65° C., followed by one or more washes at 0.2×SSC, 1% SDS at 65° C. Very high stringency conditions are the preferred conditions and the ones that should be used unless otherwise specified. Several factors are thought to influence the stringency of hybridization other than the above-described SSC concentration, and one skilled in the art can suitably select these factors to accomplish a similar stringency.

In another aspect, the invention includes an expression cassette or expression vector in which a nucleic acid mentioned above is operably linked to an expression control sequence. Such an expression vector can be used to transfect cells to thereby produce a protein or polypeptide encoded by a nucleic acid of the invention.

The invention can also include a cultured host cell or its progeny transfected with the cassette or vector and expressing the polypeptide encoded by the cassette or vector. The host cell can be prokaryotic or eukaryotic cell. Examples of such a cell include bacterial cells (such as E. coli), insect cells, yeast cells, mammalian cells, or other suitable cells.

The invention can further include a method of producing the polypeptide from a cultured host cell. In some embodiments, the method includes culturing the cell under conditions permitting expression of the polypeptide, and purifying the polypeptide from the cell or the medium of the host cell.

In another aspect, the invention includes an expression vector that contains a first nucleic acid with the sequence of SEQ ID NO:3 and a second nucleic acid sequence encoding a gene. The 5′-end of the second sequence encoding the gene is operatively linked to the 3′-end of the first sequence. In some embodiments, the gene encodes a reporter protein, such as a green fluorescent protein, a luciferase, or a lacZ. In some embodiments, the gene encodes a suicide protein, such as a toxin. In another aspect, the invention also includes a cultured cell or cell system that contains the expression vector. In some embodiments, the cell is a mammalian cell. In preferred embodiments, the cell is a multidrug-resistant cancer cell.

In another aspect, the invention features an isolated polypeptide that has an amino acid sequence at least 70% identical to the amino acid sequence of SEQ ID NO:2. In some embodiments, the polypeptide, when expressed in a drug-sensitive cell, renders the cell resistant to DNA-damaging agents.

The term “isolated polypeptide” or “purified polypeptide” as used herein in reference to a given polypeptide or protein (e.g., an antibody) means that the polypeptide or antibody is substantially free from other biological macromolecules, such as cellular material or other contaminating proteins from the cell or tissue source from which the polypeptide is derived. The polypeptide is also substantially free from chemical precursors or other chemicals when chemically synthesized. The substantially pure polypeptide or antibody is at least 75% (e.g., at least 80, 85, 95, or 99%) pure by dry weight. Purity can be measured by any appropriate standard method, for example, by column chromatography, polyacrylamide gel electrophoresis, or HPLC analysis.

In another aspect, the invention features a purified antibody that binds specifically to a polypeptide with the amino acid sequence of SEQ ID NO:2 or its fragments.

In another aspect, the invention features a nucleic acid, containing the sequence of SEQ ID NO:3, that is operably linked to a heterologous sequence. As used herein, the term “heterologous sequence” is defined as a sequence that is not naturally flanked by a nucleic acid having the sequence of SEQ ID NO:3. In some embodiments, the heterologous sequence is a coding sequence, e.g., a gene encoding a reporter protein. In some other embodiments, the heterologous sequence is a non-coding sequence, e.g., a promoter sequence.

In another aspect, the invention features a method of detecting a cellular proliferative disorder or drug-resistant cells in a subject. The method includes providing a test sample of a subject and measuring the expression level of a gene encoding a polypeptide with a sequence of SEQ ID NO:2 in the test sample. In some embodiments, the expression level of the gene is the amount of an mRNA of the MRP3s1 gene. In some embodiments, the expression level is the amount of a polypeptide with a sequence of SEQ ID NO:2. In one embodiment, the method includes contacting an antibody against the polypeptide with the test sample and detecting binding of the antibody. In some embodiments, the method also includes reporting the expression level of the MRP3s1 gene in the test sample.

Examples of cellular proliferative disorders include cancer, e.g., carcinoma, sarcoma, metastatic disorders or hematopoietic neoplastic disorders, e.g., leukemias. A metastatic cancer can arise from a multitude of primary cancer types, including but not limited to those of prostate, colon, lung, breast and liver origin.

“Subject,” as used herein, refers to human and non-human animals. “Non-human animals” of the invention includes all vertebrates, e.g., mammals, such as non-human primates (particularly higher primates), dog, rodent (e.g., mouse or rat), guinea pig, cat, and non-mammals, such as birds, amphibians, reptiles, etc. In a preferred embodiment, the subject is a human. In another embodiment, the subject is an experimental animal or animal suitable as a disease model.

A “test sample” of a subject is a cellular tissue from a mammal, preferably a human, suspected of having multidrug-resistance. The tissue can be any body tissue type, which comprises cells, including body fluid cell suspensions (e.g., blood; lymph, cerebrospinal fluid, peritoneal fluid or ascites fluid). Preferably the cellular tissue is obtained from a body tissue suspected of comprising transformed cells. Accordingly, the present method provides information relevant to diagnosis of the presence of a multidrug-resistant cancer. In some embodiments, the method includes treating a previous sample obtained from the subject at an earlier time, measuring the expression level of a gene, and reporting the expression levels in the sample and the previous sample.

“Reporting the expression level” can be carried out via any means, including: oral communication, paper documentation or reports, and electronic storing/transferring, including e-mail and Internet correspondence. In one preferred embodiment, the method consists of comparing the expression level to a predetermined value. This “predetermined value” can be the expression level of MRP3s1 gene in a previous test sample obtained from the same subject at an earlier time, or the expression level of MRP3s1 gene in a test sample of a healthy subject.

In another aspect, the invention features a method for monitoring a subject undergoing a therapeutic treatment or for determining whether a subject is a candidate for multidrug-resistance therapy. This method consists of obtaining a sample from a subject, treating the sample, and measuring the expression level of a gene encoding a polypeptide with a sequence of SEQ ID NO:2 in the sample.

In another aspect, the invention features a therapeutic method of treating or targeting a subject at risk of (or susceptible to) a cellular proliferative disorder or having a disorder associated with aberrant or unwanted expression or activity of a polypeptide encoded by a nucleic acid of the invention or with the amino acid sequence of SEQ ID NO:2. The method includes identifying a subject suffering (or at risk of) a cellular proliferative disorder and administering to the subject a therapeutic agent. Identifying a subject in need of such treatment can be in the judgment of a subject or a health care professional and can be subjective (e.g. opinion) or objective (e.g. measurable by a test or diagnostic method).

As used herein, the term “treatment” or “targeting” is defined as the application or administration of a therapeutic agent to a patient, or application or administration of a therapeutic agent to an isolated tissue or cell line from a patient, who has a disease, a symptom of disease or a predisposition toward a disease, with the purpose to cure, alleviate, alter, ameliorate, improve, or affect the disease, the symptoms of disease or the predisposition toward disease. An “effective amount” refers to an amount of the agent that is sufficient to provide a therapeutic or healthful benefit, or reducing the probability of relapse after a successful course of treatment. The term “therapeutic agent” as used herein means any molecule that binds to a polypeptide or a nucleic acid of the present invention, or any molecule that modulates the expression level of the polypeptide or nucleic acid. The therapeutic agent includes, but is not limited to, small molecules, peptides, antibodies, ribozymes, and antisense oligonucleotides. In one embodiment, an agent binds to a polypeptide with the amino acid sequence of SEQ ID NO:2 or its fragments. The binding or modulating by the agent inhibits the drug resistance activity of the polypeptide are encompassed by invention. In some embodiments, the agent is a small molecule compound that binds to the polypeptide. In some embodiments, the agent is an antibody against the polypeptide. In some embodiments, the antibody is linked to a substance whose action can destroy a cell. Examples of such substances include a radioactive isotope, a toxin, or a chemotherapeutic drug, as well as a cell whose action can destroy a cell, such as a cytotoxic cell.

In another aspect, the invention features a method of expressing in a drug-resistant cell in vivo a foreign polypeptide that can bind to a polypeptide with the amino acid sequence of SEQ ID NO:2. This method includes providing an expression vector encoding the foreign polypeptide, introducing the vector into the cell in vivo, and maintaining the cell in vivo under conditions permitting expression of the foreign polypeptide in the cell. In some embodiments, the method includes expressing an antibody or a mutant form of MRP3s1 polypeptide in the cell.

In another aspect, the invention features a method of introducing into a cell in vivo a foreign nucleic acid complementary (or antisense) to SEQ ID NO: 1 or its fragments. The method includes providing a sequence containing the foreign nucleic acid and contacting the sequence with the cell in vivo. An “antisense” nucleic acid can include a nucleotide sequence that is complementary to a sense nucleic acid encoding a polypeptide or protein, e.g., complementary to the coding strand of a double-stranded cDNA molecule or complementary to an mRNA sequence. In some embodiments, the foreign nucleic acid can be an antisense sequence of MRP3s1 nucleic acid. The antisense nucleic acid can be complementary to an entire coding strand of the MRP3s1 gene, or to only a portion thereof. In another embodiment, the antisense nucleic acid molecule is antisense to a “noncoding region” of the coding strand of a nucleotide sequence encoding MRP3s1 polypeptide, the 5′ and 3′ untranslated regions.

In another aspect, the invention features a method for targeting a cellular proliferative disorder in a subject. The method includes identifying a subject having a cellular proliferative disorder and administering to the subject an agent that can bind to a nucleic acid with the sequence of SEQ ID NO:3 or to a nucleic acid encoding a polypeptide with the amino acid sequence of SEQ ID NO:2. In some embodiments, the method includes administering an antisense sequence of MRP3s1 gene.

In another aspect, the invention features a method for targeting a cellular proliferative disorder in a subject. The method includes identifying a subject having a cellular proliferative disorder and administering to the subject an agent that can modulate the expression level of a gene encoding a polypeptide with the amino acid sequence of SEQ ID NO:2. In some embodiments, the method includes modulating the expression of the MRP3s1 gene.

In another aspect, the invention features a method of modulating the cellular pump mechanism of a resistant cancer cell. The method includes providing an agent that binds to a polypeptide with the amino acid sequence of SEQ ID NO:2 or its fragments and contacting the agent with the cell.

In another aspect, the invention features a method of modulating the cellular pump mechanism of a resistant cancer cell in a subject. The method includes administering to a subject having a resistant cancer cell an agent that binds to a polypeptide with the amino acid sequence of SEQ ID NO:2.

In another aspect, the invention features a cell system for screening for a therapeutic agent for treating a drug-resistant cancer cell. The cell system contains a reporter gene operatively linked to a regulatory sequence constructed and arranged to drive the transcription of the reporter gene. In some embodiments, the regulatory sequence contains a nucleic acid with the sequence of SEQ ID NO:3. In some embodiments, the reporter gene encodes a polypeptide with the sequence of SEQ ID NO:2. In some other embodiments, the reporter gene encodes a green fluorescent protein, a luciferase, or a lacZ. In some embodiments, the cell system is a host cell line or a host cell in a transgenic animal; the reporter gene is in a vector or in the genome of the host cell.

In another aspect, the invention features a method of screening for a therapeutic agent for treating a drug-resistant cancer cell. The method includes providing a cell system that is mentioned above, contacting the cell system with a candidate agent; and measuring the level of synthesis of the gene product of the reporter gene. A decreased level of synthesis in the presence of the candidate agent compared to in the absence of the agent is indicative of the agent being an effective agent for treating a drug-resistant cancer cell. In some embodiments, the reporter gene encodes a green fluorescent protein, a luciferase, a lacZ, or a polypeptide with the sequence of SEQ ID NO:2.

In another aspect, the invention features a method of making an antibody. The method includes immunizing a non-human animal with an immunogenic fragment of a polypeptide with the sequence of SEQ ID NO:2.

In another aspect, the invention features a method of making an antibody. The method includes providing a hybridoma cell that produces a monoclonal antibody specific for a polypeptide with the sequence of SEQ ID NO:2 and culturing the cell under conditions that permit production of the monoclonal antibody.

In another aspect, the invention features a method of modulating expression of a gene responsible for controlling cellular pump mechanisms in a cell. The method includes providing an effective amount agent that binds to a nucleic acid containing the nucleotide sequence of SEQ ID NO:3 or fragments thereof, and contacting the agent with the cell.

In yet another aspect, the invention features a method of delivering a suicide protein to a tumor cell. The method consists of (i) providing an expression vector containing a first nucleic acid with the sequence of SEQ ID NO:3 and a second nucleic acid sequence that is operatively linked to the first sequence and (ii) contacting the vector with the cell. The second sequence encodes a protein that is toxic to the cell. Examples of such a suicide protein include a polypeptide toxin, such as ricin or diphtheria toxin.

Other features or advantages of the present invention will be apparent from the following detailed description, and also from the claims.

### DESCRIPTION OF DRAWINGS

FIGS. 1A-1B are photographs of Northern blotting results showing the expression of the MRP3s1 gene in normal tissues (1A) and cancer cell lines (1B).

FIG. 2 is a photograph of western blotting results showing the expression of the MRP3s1 gene in cancer cell line HT-29 detected by different monoclonal antibodies.

FIGS. 3A and 3B are immunofluorescence microscopic photos (3A) of MCF-7 cells and MCF-7 cells transfected with an MRP3s1 expression vector and diagrams (3B) showing drug sensitivity of the cells.

FIG. 4 are sequences for MRP3s1 nucleic acid, polypeptide and promoters.

### DETAILED DESCRIPTION

The present invention relates generally to drug-resistant cancers. The invention features genes (e.g., the MRP3s1 gene), promoter sequences of the genes, and polypeptides, (e.g., the MRP3s1 polypeptide), involved in multidrug-resistance. The invention further features diagnosis of multidrug-resistance cancer, monitoring of the efficacy of a chemotherapeutic regimen, and design of novel chemotherapeutic drugs that are cytotoxic to cells expressing the MRP3s1 gene.

Isolated Nucleic Acids

In one aspect, the invention provides an isolated nucleic acid that contains a nucleotide sequence at least 70% (e.g., 70%, 80%, 90%, 95% . . . etc.) identical to the sequence of SEQ ID NO:3. The invention also provides an isolated nucleic acid encoding a polypeptide with biological activity of MRP3s1. In one aspect, the nucleic acid encoding a polypeptide that is at least 70% (e.g., 70%, 80%, 90%, 95% . . . etc.) identical to amino acid sequence of SEQ ID NO:2. In another aspect, the nucleic acid encodes a polypeptide having an amino acid sequence of SEQ ID NO:2. In another aspect, the nucleic acid is a cDNA comprising a nucleotide sequence SEQ ID NO:1 or its degenerate variants. In yet another aspect, invention provides an isolated nucleic acid having a sequence that, under low, medium or high stringency conditions, hybridizes to a hybridization probe with the sequence of SEQ ID NO:1 or SEQ ID NO:3, or their complements. In some embodiments, the expression of a polypeptide encoded by SEQ ID NO:1 in a drug sensitive cell renders the cell resistant to cytotoxic drugs, e.g., a DNA-damaging agent.

A nucleic acid of the invention can be isolated from multidrug-resistant cancer cells by preparing a cDNA library from these cells using standard techniques, and screening this library with cDNA produced from total mRNA isolated from a multidrug-resistant cell and a drug sensitive cell. For example, a cDNA library from drug-resistant breast cancer cells is prepared. The library is plated on two sets of replica filters by standard methods. One set of filters is then screened with cDNA prepared from a cisplatin-resistant breast cancer cell line (e.g., MCF-7/CDDP) and the other set of filters is screened with a comparable amount of cDNA prepared from a cisplatin-sensitive cell line (e.g., MCF-7). The cDNA used for screening the library is labelled, typically with a radioactive isotope, such as ³²P. Following visualization of the hybridization results by standard procedures, cDNA clones displaying increased hybridization with MCF-7/CDDP cDNA when compared to MCF-7 cDNA can be selected from the library. These cDNA clones represent mRNAs overexpressed in MCF-7/CDDP cells when compared with MCF-7 cells. Alternatively, a nucleic acid of the present invention can be isolated from an expression library from a cancer cell using antibodies provided in this invention. An example is delineated in the Example section below.

Determination of whether a nucleic acid so isolated or isolated in the manner described below encodes a polypeptide having the biological activity of MRP3s1 can be accomplished by expressing the nucleic acid in a non-multidrug-resistant mammalian cell, according to standard techniques known in the art including those described herein, and assessing whether the expression of the nucleic acid in the cell confers on the cell multidrug-resistance to drugs, such as cisplatin. A nucleic acid encoding a polypeptide having the biological activity of MR3s1 can be sequenced by standard techniques, such as dideoxynucleotide chain termination, to determine the nucleic acid sequence and predict amino acid sequence of the encoded polypeptide.

A nucleic acid of the invention can also be isolated by preparing a labelled nucleic acid probe having all or part of the nucleotide sequence of SEQ ID NO:1 or SEQ ID NO:3 and using this labelled nucleic acid probe to screen an appropriate DNA library (e.g. a cDNA or genomic DNA library). For instance, a cDNA library made from a multi-drug resistant cell line as described above can be used to isolate a cDNA encoding a polypeptide having MRP3s1 activity by screening the library with the labelled probe using standard techniques. A genomic DNA library can be similarly screened to isolate a genomic clone encompassing a regulatory region or a gene encoding a polypeptide having the activity. Nucleic acids isolated by screening of a cDNA or genomic DNA library can be sequenced by standard techniques.

A nucleic acid of the invention can also be isolated by selectively amplifying a nucleic acid encoding a polypeptide with MRP3s1 activity using the polymerase chain reaction (PCR) method and genomic DNA or mRNA. To prepare cDNA from mRNA, total cellular mRNA can be isolated, for instance from a multidrug-resistant cell line, by a variety of techniques, e.g., by using the guanidinium-thiocyanate extraction procedure of Chirgwin et al., Biochemistry, 18:5294-5299, 1979. cDNA is then synthesized from the mRNA using reverse transcriptase. Moloney MLV reverse transcriptase, such as those available from Gibco/BRL, Bethesda, Md. Synthetic oligonucleotide primers can be designed according to the nucleotide sequence of SEQ ID NO:1 or SEQ ID NO:3. Using these oligonucleotide primers and standard PCR amplification technique, a nucleic acid can be amplified from cDNA or genomic DNA. The amplified nucleic acid can be cloned into an appropriate vector and characterized by DNA sequence analysis.

A nucleic acid of the invention can also be chemically synthesized using standard techniques. Various methods for chemically synthesizing polydeoxynucleotides can be found in e.g., Itakura et al., U.S. Pat. No. 4,598,049 and Caruthers et al., U.S. Pat. No. 4,458,066.

A nucleic acid of the invention can also be identified according to high homology (e.g., at least 70% identity) between a nucleic acid and SEQ ID NO:1 or SEQ ID NO:3. Similarly, a nucleic acid can be identified based on a high homology between SEQ ID NO:2 and a polypeptide encoded by the nucleic acid. Such homology or percent identity of two nucleic acids or of two amino acid sequences can be determined using the algorithm of Karlin and Altschul (Proc. Natl. Acad. Sci. USA 87:2264-2268, 1990), modified as in Karlin and Altschul (Proc. Natl. Acad. Sci. USA 90:5873-5877, 1993).

Further, non-coding sequences of a nucleic acid of the invention can be characterized. For example, the intron-exon structure and the transcription regulatory sequences of the gene encoding the MRP3s1 polypeptide can be identified by using a nucleic acid of the invention to probe a genomic DNA clone library. Regulatory elements, such as promoter and enhancers necessary for expression of the gene in various tissues, can be identified using conventional techniques. The function of the elements can be confirmed by using them to express a reporter gene, such as the genes of lacZ, luciferase, or green fluorescent protein, that is operatively linked to the elements. Such a construct can be introduced into cultured cells using standard procedures or into non-human transgenic animal models. In addition to identifying regulatory elements, such constructs can be used to identify proteins interacting with the elements and molecules modulating the expression of a nucleic acid of the invention using techniques known in the art.

Isolated/Purified Polypeptides

An isolated polypeptide of the invention has an amino acid sequence at least 70% (e.g., 70%, 80%, 90%, 95% . . . etc.) identical to the amino acid sequence of SEQ ID NO:2. In some embodiments, the polypeptide, when expressed in a drug-sensitive cell, renders the cell resistant to DNA-damaging agents. Immunogenic portions of polypeptides are within the scope of the invention.

A polypeptide of the invention can be prepared by expressing an above-described nucleic acid in a suitable host cell and isolating the polypeptide encoded by the nucleic acid using techniques known in the art. The invention provides a method of preparing an isolated polypeptide. The method includes introducing into a host cell a recombinant nucleic acid encoding the polypeptide, allowing the polypeptide to be expressed in the host cell, and isolating the polypeptide. Preferably, the recombinant nucleic acid is a recombinant expression vector, which is described below. A polypeptide can be isolated from a host cell expressing it according to standard procedures of the art, including ammonium sulfate precipitation and fractionation column chromatography (e.g., ion exchange, gel filtration, electrophoresis, affinity chromatography, etc.).

Expression Vectors

A nucleic acid of the invention can be incorporated into a recombinant expression vector, where the nucleic acid or its fragment is operatively linked to a regulatory sequence suitable for a host cell to be used for expression. Examples of such a regulatory sequence include promoters, enhancers and other expression control elements that are known to those skilled in the art. Such expression vectors can be used to transfect host cells to thereby produce a protein or polypeptide encoded by a nucleic acid of the invention. Note that the design of the expression vector may depend on factors, such as the choice of the host cell and/or the type of polypeptide to be expressed. The recombinant expression vectors of the invention can be designed for expression of polypeptides in prokaryotic or eukaryotic cells. For example, polypeptides can be expressed in bacterial cells such as E. coli, insect cells (using baculovirus), yeast cells, mammalian cells, or other suitable host cells.

The recombinant expression vector can have a nucleic acid of the invention cloned into the expression vector in an antisense orientation. That is, the nucleic acid is operatively linked to a regulatory sequence in a manner that allows for expression of an RNA molecule that is antisense or complement to the nucleotide sequence of SEQ ID NO:1. Regulatory sequences operatively linked to the antisense nucleic acid can be chosen, which direct the continuous expression of the antisense RNA molecule in a variety of cell types, for instance, a viral promoter and/or enhancer, or regulatory sequences can be chosen, which direct tissue or cell type specific expression of an antisense RNA, as described above.

A nucleic acid of the invention that does not encode a polypeptide, e.g., SEQ ID NO:3, can be incorporated into a recombinant expression vector. Such a nucleic acid can be operatively linked to heterologous sequence. This heterologous sequence can be a coding sequence, e.g., a gene encoding a reporter protein including the lacZ or the green fluorescent protein. This heterologous sequence can also be a non-coding sequence, e.g., a promoter sequence.

Host Cells

The recombinant expression vectors of the invention can be used to make a host cell having the recombinant expression vector. Examples of the host cell include a prokaryotic and eukaryotic cell that have been transformed or transfected with a recombinant expression vector of the invention. A prokaryotic cell can be transformed with nucleic acid (e.g., SEQ ID NO:1) by electroporation. The nucleic acid can be introduced into a mammalian cell via conventional techniques such as calcium phosphate co-precipitation, DEAE-dextran-mediated transfection, lipofectin, electroporation, microinjection, or virus-mediated methods. Suitable methods for transforming and transfecting a cell can be found in, e.g., Sambrook et al. (Molecular Cloning: A Laboratory Manual, 2nd Edition, Cold Spring Harbor Laboratory press (1989)).

A nucleic acid introduced into a mammalian cell can be integrated into the genome of the cell. To identify such an integrant or stable transfectant, a gene that contains a selectable marker (e.g., resistance to antibiotics) can be introduced into a cell along with the nucleic acid of interest. Preferred selectable markers include those conferring resistance to certain drugs, such as G418 and hygromycin. Selectable markers can be introduced on a separate vector from the nucleic acid of interest or, preferably, on a same vector. A host cell transformed with one or more recombinant expression vectors containing a nucleic acid of the invention and a gene for a selectable marker can be identified by selecting for a cell using the selectable marker.

Antibodies

A polypeptide of the invention or its fragment can be used as an immunogen in an immunization preparation to generate antibodies specific for the polypeptide according to conventional methods. More specifically, a mammal (e.g., a mouse, rat, or rabbit) can be immunized with an immunogenic form of the polypeptide (e.g., isolated polypeptide, recombinantly produced protein, or synthetic peptide) that elicits an antibody response in the mammal. Alternatively, a mammal can be immunized with a cell that expresses the polypeptide or a membrane fraction of the cell. An immunization preparation should contain an effective immunogenic amount of the polypeptide, which can optionally be conjugate linked to a carrier. The effective amount of immunogen per unit dose depends, among other things, on the species of animal inoculated, the body weight of the animal, and the chosen immunization regimen, as are well known in the art. An immunization preparation can also include an adjuvant, such as complete Freund's adjuvant or incomplete Freund's adjuvant. The progress of immunization can be monitored by detection of antibody titers in plasma or serum. Standard enzyme linked immunosorbent assays (ELISAs), immunoprecipitations, immunofluorescence, enzyme immunoassay (EIA), radioimmunoassay (RIA), Western Blot, or other immunoassays can be used with the immunogen as antigen to assess the level of antibody titers. Following immunization, antisera can be obtained and, if desired, polyclonal antibodies can be isolated from the sera.

To produce monoclonal antibodies, antibody-producing lymphocytes can be harvested from an immunized animal and fused with myeloma cells by standard somatic cell fusion procedures. The resulting hybridoma cells can be screened immunochemically for production of antibodies specifically reactive with a polypeptide and monoclonal antibodies isolated by standard techniques. Detailed procedures for making a monoclonal antibody can be found in, e.g., Harlow E et al., 1988, Antibodies: A Laboratory Manual, Cold Spring Harbor Laboratory Press, Cold Spring Harbor, N.Y. An example of making hybridomas producing monoclonal antibodies against MRP3s1 will be described in detail in Example 6 below.

Antibodies against a polypeptide of this invention can also be generated by the genetic immunization methods as essentially described in, e.g., Barry, M. et al., Biotechniques 16:616-620, 1994 and Krasemann S. et al., J. Biotechnol. 73: 119-129, 1999, and in chicken egg-yolk, bacteria, bioreactors, or plants by the methods described in Tini M. et al., Comp Biochem Physiol A Mol Integr Physiol. 131:569-74, 2002, Ward E. FASEB J. 6:2422-7, 1992, Skerra A. Curr Opin Immunol. 5:256-62, 1993, Fischer R. et al., J Biol Regul Homeost Agents. 14:83-92, 2000, and Falkenberg F. Res Immunol. 149:560-70, 1998.

The antibodies generated in the manner described above can be used to quantify the amount of a polypeptide of the invention or its fragments to diagnose multidrug-resistance and to determine the role of the polypeptide in particular cellular events or pathological states, particularly its role in multidrug-resistance. For example, the antibodies can be used to detect a polypeptide of the invention or its fragments in various biological materials using ELISA, radioimmunoassay, or histochemical techniques.

The antibodies can be physically linked to a detectable substance. Suitable detectable substances include various enzymes, bridging complexes, fluorescent materials, luminescent materials and radioactive materials. Examples of suitable enzymes include horseradish peroxidase, alkaline phosphatase, beta-galactosidase, or acetylcholinesterase; examples of suitable bridging complexes include avidin/biotin and Protein A/antibody; examples of suitable fluorescent materials include fluorescein or rhodamine; an example of a luminescent material includes luminol; and examples of suitable radioactive materials include ¹⁴C, ³H, ¹²⁵H, or ³⁵S.

The antibodies can also be linked to a substance having toxic or therapeutic activity. The “substance having toxic or therapeutic activity” includes molecules whose action can destroy a cell, such as a radioactive isotope or a toxin (e.g., diphtheria toxin). Examples of the substances also include chemotherapeutic agents such as carboplatin and methotrexate. Preferably, the chemotherapeutic agent is not a drug to which a polypeptide of the invention confers resistance. As the antibodies can be bound to Fc receptors on cytotoxic cells, examples of the substance also include cytotoxic cells such as macrophages, neutrophils, eosinophils, NK cells, LAK cells, and large granular lymphocytes.

One can also make a bispecific antibody that binds to a polypeptide of the invention and a second molecule. A bispecific antibody-producing hybridomas can be prepared using the procedures known in the art, e.g., those disclosed in Staerz et al., Proc. Natl. Acad. Sci. USA 83: 1453, 1986 and Immunology Today 7:241, 1986. To prepare such a bispecific antibody, a hybridoma is formed by fusing a first cell line producing a first monoclonal antibody capable of binding to a polypeptide of the invention and a second cell line producing a second monoclonal antibody capable of binding to a second molecule. This second molecule can be a detectable substance, or a substance having toxic or therapeutic activity.

One can also make a tetrameric antibody complex using the method described in U.S. Pat. No. 4,868,109. More specifically, one can make the complex by preparing a first monoclonal antibody capable of binding to a polypeptide of the invention and a second monoclonal antibody capable of binding to a second molecule, such as detectable substance or a substance having toxic or therapeutic activity. The first and the second antibody are from a first animal species and the Fc-fragments of both antibodies can be bound by a third antibody from a second animal species. Therefore, the antibodies can form a complex.

The above-mentioned antibodies, bispecific antibodies, and tetrameric antibody complexes can be used to treat multidrug-resistant cancers. A composition containing antibodies, bispecific antibodies or tetrameric anti body complexes in a pharmaceutically acceptable carrier can be administered to a subject in need. Preferably, the antibodies, bispecific antibodies or tetrameric antibody complexes are coupled to or capable of binding to a substance having toxic or therapeutic activity and to a cancer cell expressing a polypeptide of the invention.

Diagnostic and Prognostic Assays

A multidrug-resistant cancer cell can be detected in a subject based on the presence of a polypeptide or a nucleic acid (e.g., mRNA) encoding the polypeptide in a test sample from the subject. In other words, the polypeptide and nucleic acids can be used as markers to indicate the presence or absence of a multidrug-resistant cancer cell. Diagnostic and prognostic assays of the invention include methods for assessing the expression level of MRP3s1 polypeptide or nucleic acid and for identifying variations and mutations in the sequence of MRP3s1 polypeptide or nucleic acid.

The presence, level, or absence of MRP3s1 polypeptide or nucleic acid in a test sample can be evaluated by obtaining a test sample from a test subject and contacting the test sample with a compound or an agent capable of detecting MRP3s1 polypeptide or nucleic acid (e.g., mRNA or genomic DNA). The “test sample” includes tissues, cells and biological fluids isolated from a subject, as well as tissues, cells and fluids present within a subject. The level of expression of the MRP3s1 gene can be measured in a number of ways, including measuring the mRNA encoded by the MRP3s1 gene; measuring the amount of polypeptide encoded by the MRP3s1 gene; or measuring the activity of polypeptide encoded by the MRP3s1 gene.

The level of mRNA corresponding to the MRP3s1 gene in a cell can be determined both by in situ and by in vitro formats. mRNA isolated from a test sample can be used in hybridization or amplification assays that include, Southern or Northern analyses, PCR analyses, and probe arrays. One preferred diagnostic method for the detection of mRNA levels involves contacting the isolated mRNA with a nucleic acid probe that can hybridize to the mRNA encoded by the MRP3s1 gene. The probe can be a full-length MRP3s1 nucleic acid, such as the nucleic acid of SEQ ID NO:1, or a portion thereof, such as an oligonucleotide of at least 10 nucleotides in length and sufficient to specifically hybridize under stringent conditions to MRP3s1 mRNA or genomic DNA.

In one format, mRNA (or cDNA prepared from it) is immobilized on a surface and contacted with the probes, for example, by running the isolated mRNA on an agarose gel and transferring the mRNA from the gel to a membrane, such as nitrocellulose. In another format, the probes are immobilized on a surface and the mRNA (or cDNA) is contacted with the probes, for example, in a gene chip array. A skilled artisan can adapt known mRNA detection methods for use in detecting the level of mRNA encoded by the MRP3s1 gene.

The level of mRNA (or cDNA prepared from it) in a sample encoded by MRP3s1 gene can be evaluated with nucleic acid amplification, e.g., by standard PCR (U.S. Pat. No. 4,683,202), RT-PCR (Bustin S. J Mol Endocrinol. 25:169-93, 2000 and Freeman W. et al., Biotechniques. 26:112-22, 124-5, 1999), quantitative PCR (Ong Y. et al., Hematology. 7:59-67, 2002 and Jung R. et al., Clin Chem Lab Med. 38:833-6, 2000), real time PCR (Ginzinger D. Exp Hematol. 30:503-12, 2002), and in situ PCR (Thaker V. Methods Mol Biol. 115:379-402, 1999, and Muro-Cacho C. Front Biosci. 2:c15-2, 1997), or any other nucleic acid amplification method, followed by the detection of the amplified molecules using techniques known in the art. As used herein, amplification primers are defined as being a pair of nucleic acid molecules that can anneal to 5′ or 3′ regions of a gene (plus and minus strands, respectively, or vice-versa) and contain a short region in between; Under appropriate conditions and with appropriate reagents, such primers permit the amplification of a nucleic acid molecule comprising the nucleotide sequence flanked by the primers.

For in situ methods, a cell or tissue sample can be prepared and immobilized on a support, such as a glass slide, and then contacted with a probe that can hybridize to mRNA that encodes the MRP3s1 polypeptide.

In another embodiment, the methods of the invention further include contacting a control sample with a compound or agent capable of detecting MRP3s1 mRNA, or genomic DNA, and comparing the presence of MR3s1 mRNA or genomic DNA in the control sample with the presence of MRP3s1 mRNA or genomic DNA in the test sample.

The above-described nucleic acid-based diagnostic methods can provide qualitative and quantitative information to determine whether a subject has or is predisposed to a disease associated with aberrant MRP3s1 gene expression, e.g., multidrug-resistance cancers.

A variety of methods can be used to determine the level of MRP3s1 polypeptide. In general, these methods include contacting an agent that selectively binds to the polypeptide, such as an antibody, to evaluate the level of polypeptide in a sample. Antibodies can be polyclonal, or more preferably, monoclonal. An intact antibody, or a fragment thereof (e.g., Fab or F(ab′)₂) can also be used. In a preferred embodiment, the antibody bears a detectable label. The term “labeled”, with regard to the probe or antibody, is intended to encompass direct labeling of the probe or antibody by physically linking a detectable substance to the probe or antibody, as well as indirect labeling of the probe or antibody by reactivity with a detectable substance. For example, an antibody with a rabbit Fc region can be indirectly labelled using a second antibody directed against the rabbit Fc region, wherein the second antibody is coupled to a detectable substance. Examples of detectable substances are provided herein.

The detection methods can be used to detect MRP3s1 polypeptide in a test sample in vitro as well as in vivo. In vitro techniques for detection of MRP3s1 polypeptide include ELISAS, immunoprecipitations, immunofluorescence, EIA, RIA, and Western blot analysis. In vivo techniques for detection of MRP3s1 polypeptide include introducing into a subject a labeled anti-MRP3s1 antibody. For example, the antibody can be labeled with a detectable substance as described above. The presence and location of the detectable substance in a subject can be detected by standard imaging techniques.

The diagnostic methods described herein can identify subjects having, or at risk of developing, a disease or disorder associated with misexpressed or aberrant or unwanted MRP3s1 expression or activity. As used herein, the term “unwanted activity” includes an unwanted phenomenon involved in a biological response such as multidrug-resistance or deregulated cell proliferation. In one embodiment, a disease or disorder associated with aberrant or unwanted MRP3s1 expression or activity is identified. A test sample is obtained from a subject and the MRP3s1 polypeptide or nucleic acid (e.g., mRNA) is evaluated. The level of MRP3s1 polypeptide or nucleic acid is diagnostic for a subject having or at risk of developing a disease or disorder associated with aberrant or unwanted MRP3s1 expression or activity. In order to prevent samples from being degraded, the samples can be stored at temperatures below −20° C. A tissue section, for example, a freeze-dried or fresh frozen section of cancer tissue removed from a subject, can also be used as the sample. The samples can be fixed and the appropriate method of fixation is chosen depending upon the type of labelling used for the antibodies.

The prognostic assays described herein can be used to determine whether a subject is suitable to be administered with an agent (e.g., an agonist, antagonist, peptidomimetic, protein, peptide, nucleic acid, small molecule, or other drug candidate) to treat a disease or disorder associated with aberrant or unwanted MRP3s1 expression or activity. For example, such methods can be used to determine whether a subject can be administered with a cytotoxic drug to treat a cell proliferation disorder.

Also featured is a method of evaluating a sample. The method includes providing a test sample, e.g., from a subject, and determining a gene expression level in the test sample. The level includes a value representing the expression level of MRP3s1, which can be determined by any of the methods described herein. The method can further include comparing the value to a predetermined value. The method can be used to diagnose a multidrug-resistant cancer in a subject and an increase in MRP3s1 expression is an indication that the subject has a multidrug-resistant cancer. The method can also be used to monitor a treatment for a multidrug-resistant cancer in a subject. For this purpose, gene expression levels can be determined for test samples from a subject before, during, or after undergoing a treatment. An increase of the expression level of MRP3s1 after the treatment indicates that the subject has developed resistance to the treatment.

Information obtained from practice of the above diagnostic assays is expected to be useful in prognostication, identifying progression of, and clinical management of diseases and other deleterious conditions affecting an individual's health status. In preferred embodiments, the foregoing diagnostic assays provide information useful in prognostication, identifying progression of and management of malignancies (cancers) that are characterized by expression of MRP3s1 and thus by a multidrug-resistance phenotype. The information more specifically assists the clinician in designing chemotherapeutic or other treatment regimes to eradicate such malignancies from the body of an afflicted mammal, typically a human.

Screening Assays

The invention provides screening assays for identifying test or candidate compounds or agents (e.g., peptides or small molecules) that bind to a polypeptide encoded by a nucleic acid of this invention, e.g., an MRP3s1 polypeptide. These compounds (or agents) can have stimulatory or inhibitory effects on the expression or activity of the polypeptide. Compounds thus identified can be used to modulate the activity of the polypeptide in a therapeutic protocol, or to elaborate the biological function of the polypeptide.

In one embodiment, the invention provides an assay for screening test or candidate compounds to identify those that bind to or modulate an activity of an MRP3s1 polypeptide or a biologically active portion thereof. The test compounds of the present invention can be obtained using any of the numerous approaches in combinatorial library methods known in the art, including: biological libraries; peptoid or peptidomimetic libraries (libraries of molecules having the biological functionalities or activities of peptides, but with a non-peptide backbone which convey desirable properties to the molecule, e.g., bioavailability, solubility, or resistance to enzymatic degradation); spatially addressable parallel solid phase or solution phase libraries (e.g., a library in multi-well devices or matrix arrays); synthetic library methods requiring deconvolution; and synthetic library methods using affinity chromatography selection. The biological library and peptoid library approaches are limited to peptide libraries, while the other approaches are applicable to peptide, non-peptide, oligomer, or small molecule libraries of compounds (Lam Anticancer Drug Des. 12:145, 1997).

In one embodiment, an assay is a cell-based assay in which a cell that expresses an MRP3s1 polypeptide or biologically active portion thereof is contacted with a test compound, and its ability to modulate MRP3s1 activity is determined. Determining the ability to modulate MRP3s1 activity can be accomplished by monitoring, for example, a resistance to Cisplatin. The cell can be of mammalian origin, e.g., human.

One can also evaluate the ability of a test compound to bind to an MRP3s1 polypeptide or to modulate the MRP3s1 polypeptide's binding to a second compound, e.g., an MRP3s1 substrate. This can be accomplished, for example, by coupling the second compound with a radioisotope or enzymatic label such that binding of the second compound to the polypeptide can be determined by detecting the labeled compound in a complex. Alternatively, the polypeptide could be coupled with a radioisotope or enzymatic label to monitor the ability of a test compound to modulate the polypeptide's binding to the second compound in a complex. The radioisotope can be detected by direct counting of radioeemnission or by scintillation counting. The enzymatic label can be horseradish peroxidase, alkaline phosphatase, or luciferase and can be detected by determination of conversion of an appropriate substrate to product.

A polypeptide encoded by a nucleic acid of the invention can interact in vivo with one or more cellular macromolecules, such as proteins. Such macromolecules are referred to as “binding partners.” Compounds that disrupt such interactions can be useful in regulating the activity of a polypeptide encoded by a nucleic acid of the invention. Such compounds can include, but are not limited to, antibodies, peptides, and small molecules. To identify compounds that interfere with the interaction between the polypeptide and its binding partner, a reaction mixture containing the polypeptide and the binding partner is prepared, under conditions and for a time sufficient, to allow them to form a complex. The reaction mixture is provided in the presence or absence of the test compound. The test compound can be initially included in the reaction mixture, or can be added at a time subsequent to the addition of the polypeptide and its binding partner. Control reaction mixtures are incubated without the test compound or with a control compound. The formation of a complex between the polypeptide and its binding partner is then detected. The formation of a complex in the control reaction, but not in the reaction mixture containing the test compound, indicates that the test compound interferes with the interaction of the polypeptide and the binding partner. A polypeptide encoded by a nucleic acid delineated herein, such as the MRP3s1 polypeptide, can be used as a “bait protein” in a two-hybrid assay or three-hybrid assay (see, e.g., Zervos et al., Cell 72:223-232, 1993) to identify binding partners. Such binding partners can be involved in regulating MRP3s1 activity as activators or inhibitors of MRP3s1 polypeptide.

In another embodiment, a test compound that modulates the expression of MRP3s1 is identified. For example, a cell expressing MRP3s1 is contacted with a test compound and the expression of MRP3s1 mRNA or polypeptide evaluated relative to the level of expression of MRP3s1 mRNA or polypeptide in the absence of the test compound. When expression of MRP3s1 mRNA or polypeptide is greater in the presence of the test compound than in its absence, the test compound is identified as a stimulator of MRP3s1 expression. Alternatively, when expression of MRP3s1 is less (statistically significantly less) in the presence of the test compound than in its absence, the test compound is identified as an inhibitor of MRP3s1 expression. The level of MRP3s1 mRNA or polypeptide expression can be determined by methods described herein for detecting MRP3s1 mRNA or polypeptide.

This invention further pertains to novel compounds identified by the above-described screening assays. Accordingly, it is within the scope of this invention to further use a compound identified as described herein (e.g., an MRP3s1 modulating agent, an antisense MRP3s1 nucleic acid molecule, an MRP3s1-specific antibody, or an MRP3s1-binding partner) in an appropriate animal model to determine the efficacy, toxicity, side effects, or mechanism of action, of treatment with such a compound. Furthermore, novel compounds identified by the above-described screening assays can be used for treatments as described herein.

Pharmaceutical Compositions

The present invention also encompasses a pharmaceutical composition comprising a compound (including a nucleic acid, peptide, and antibody of the invention) that inhibits the drug-resistance activity of the polypeptide of the invention and a pharmaceutical composition comprising a compound that inhibits the expression of a polypeptide encoded by a nucleic acid of the invention. Preferably a pharmaceutical composition is an anti-cancer agent.

When using a compound obtained by the screening method of this invention as a drug for humans and mammals, for example, cats, dogs, and horses, the compound itself can be directly administered to the patient or animal, or be given after formulating by using commonly known pharmaceutical preparation methods. For example, according to the need, the compound can be taken orally as sugarcoated tablets, capsules, elixirs, and microcapsules, or parenterally in the form of injections of aseptic solutions or suspensions with water or any other pharmaceutically acceptable liquid. The compound may be formulated by mixing with, for example, pharmacologically acceptable carriers or media, specifically, sterilized water, physiological saline, plant oils, emulsifiers, suspending agents, surfactants, stabilizers, flavoring agents, excipients, vehicles, preservatives, binders, and so on, in a unit dose form required for generally accepted drug implementation. The amount of active ingredients in these preparations makes a suitable dosage within the indicated range acquirable.

Examples for additives which can be mixed to tablets and capsules are, binders such as gelatin, corn starch, tragacanth gum, and arabic gum; excipients such as crystalline cellulose; swelling agents such as corn starch, gelatin, and alginic acid; lubricators such as magnesium stearate; sweeteners such as sucrose, lactose, or saccharin; and flavoring agents such as peppermint, Gaultheria adenothrix oil, and cherry. When the unit dosage form is a capsule, a liquid carrier, such as oil, can also be included in the above ingredients. Sterile compositions for injections can be formulated following normal drug implementations using vehicles such as distilled water used for injections.

Physiological saline and isotonic liquids including glucose or other adjuvants, such as D-sorbitol, D-mannose, D-mannitol, and sodium chloride, can be used as aqueous solutions for injections. These can be used in conjunction with suitable solubilizers, such as alcohol, specifically ethanol, polyalcohols such as propylene glycol and polyethylene glycol, non-ionic surfactants, such as polysorbate 80 (TM) and HCO-50.

Sesame oil or soy-bean oil can be used as a oleaginous liquid and may be used in conjunction with benzyl benzoate or benzyl alcohol as a solubilizer; may be formulated with a buffer such as phosphate buffer and sodium acetate buffer; a pain-killer such as procaine hydrochloride; a stabilizer such as benzyl alcohol and phenol; or an anti-oxidant. The prepared injection is filled into a suitable ampule.

The administration to patients is done by methods commonly known to those skilled in the art, such as by intra-arterial, intravenous, or subcutaneous injections, and in addition, as intranasal, bronchial, intramuscular, percutaneous, or oral administrations. One skilled in the art can also suitably select the dosage according to the body-weight or age of a patient, or the method of administration. The dosage of a compound is, generally, about 0.1 mg to about 100 mg per day, preferably about 1.0 mg to about 50 mg, more preferably, about 1.0 mg to about 20 mg, for an adult (weight 60 kg) when the compound is given orally. When administering parenterally, although there are some differences according to the patient, target organ, symptoms, and method of administration, when giving in the form of an injection to a normal adult, it is convenient to intravenously inject a dose of about 0.01 mg/kg body weight to about 1000 mg/kg body weight per day, preferably about 0.02 mg/kg body weight to about 500 mg/kg body weight per day, and more preferably about 0.05 mg/kg body weight to about 200 mg/kg body weight per day. Also, in the case of other animals, it is possible to administer an amount converted to body-weight or body-surface area.

When using a polypeptide or antibody as a drug, a therapeutically effective amount (i.e., an effective dosage) ranges from about 0.001 to 30 mg/kg body weight, preferably about 0.01 to 25 mg/kg body weight, more preferably about 0.1 to 20 mg/kg body weight. For antibodies, the more preferred dosage is generally 10 mg/kg to 20 mg/kg body weight. Modifications such as lipidation can be used to stabilize antibodies and to enhance uptake and tissue penetration. A method for lipidation of antibodies is described by Cruikshank et al. J. Acquired Immune Deficiency Syndromes and Human Retrovirology 14:193, 1997. Treatment of a subject with a protein, polypeptide, or antibody can include a single treatment or, preferably, can include a series of treatments.

If an active ingredient of a pharmaceutical composition is a nucleic acid molecule, the composition can be used for gene therapy. The nucleic acid molecule can be inserted into vectors. The resultant gene therapy vectors can be delivered to a subject by, for example, intravenous injection, local administration (see U.S. Pat. No. 5,328,470) or by stereotactic injection (see e.g., Chen et al. Proc. Natl. Acad. Sci. USA 91:3054-3057, 1994). The pharmaceutical composition can include the gene therapy vector in an acceptable diluent, or can comprise a slow release matrix in which the gene therapy vehicle is imbedded. Alternatively, where the complete gene therapy vector can be produced intact from recombinant cells, e.g., retroviral vectors, the pharmaceutical composition can include one or more cells which produce the gene delivery system.

Methods of Treatment

The present invention provides for therapeutic methods of treating or targeting a subject at risk of (or susceptible to) a disorder or having a disorder associated with aberrant or unwanted MRP3s1 expression or activity. The methods include the administration of a therapeutic agent to the subject, or to an isolated tissue or cell from the subject. Examples of the therapeutic agent include, but are not limited to, small molecules, peptides, antibodies, nucleic acid, ribozymes, and antisense oligonucleotides.

A successful treatment of cellular proliferative disorders, such as inhibiting the multidrug-resistance of a cancer cell, can be achieved by killing a cell expressing a polypeptide with an amino acid of SEQ ID NO:2. The treatment, for example, includes expressing a suicide protein in the cell using the promoter of MRP3s1 (i.e., SEQ ID NO:3). The suicide protein is operably linked to the promoter and is expressed only in the cancer cell. Accordingly, this treatment specifically targets the cancer cell expressing the polypeptide with the sequence of SEQ ID NO:2. Examples of such a suicicide protein include, but are not restricted to, herpes simplex virus-thymidine kinase (Pantuck A. et al., Hum Gene Ther, 13:777-89, 2002), bacterial cytosine deaminases (Liu J. et al., Int J Oncol. 21:661-6, 2002), and bacterial nitroreductase enzyme (Plumb J. et al., Oncogene 20(53):7797-803, 2001.)

A successful treatment of cellular proliferative disorders can also be brought about by techniques that serve to inhibit the expression or activity of a polypeptide with an amino acid of SEQ ID NO:2 in the cell. As described above, antisense and ribozyme molecules that inhibit expression of the polypeptide can be used to reduce the expression of the polypeptide. Further, molecules, e.g., an agent identified from the screening assays described above, that proves to exhibit negative modulatory activity, can be used in accordance with the invention to prevent and/or ameliorate symptoms of the disorders. Such molecules can include, but are not limited to peptides, phosphopeptides, small organic or inorganic molecules, or antibodies (including, for example, polyclonal, monoclonal, humanized, anti-idiotypic, chimeric or single chain antibodies, and Fab, F(ab′)₂ and Fab expression library fragments, and epitope-binding fragments thereof).

As described in the “Antibodies” section above, antibodies specific for a polypeptide encoded by a nucleic acid of the invention can be used to reduce activity of the polypeptide or to kill a multidrug-resistant cancer cell expressing the polypeptide. Killing of the cell can be accomplished by linking the molecule with a substance having toxic or therapeutic activity as described above.

In instances where the target antigen is intracellular and whole antibodies are used, internalizing antibodies may be preferred. Lipofectin or liposomes can be used to deliver the antibody or a fragment of the Fab region that binds to the target antigen into cells. Where fragments of the antibody are used, the smallest inhibitory fragment that binds to the target antigen is preferred. For example, peptides having an amino acid sequence corresponding to the Fv region of the antibody can be used. Alternatively, single chain neutralizing antibodies that bind to intracellular target antigens can also be administered. Such single chain antibodies can be administered, for example, by expressing nucleotide sequences encoding single-chain antibodies within the target cell population (see e.g., Marasco et al., Proc. Natl. Acad. Sci. USA 90:7889-7893, 1993.)

The molecules described above can be administered to a patient at therapeutically effective doses to prevent, treat or ameliorate disorders, such as multidrug-resistant cancers. A cancer or tumor can be any neoplastic disorder, including carcinomas, sarcomas and carcino-sarcomas. Specific types of cancer include, but are not limited to, glioma, gliosarcoma, anaplastic astrocytoma, medulloblastoma, lung cancer, small cell lung carcinoma, cervical carcinoma, colon cancer, rectal cancer, chordoma, throat cancer, Kaposi's sarcoma, lymphangiosarcoma, lymphangioendotheliosarcoma, colorectal cancer, endometrium cancer, ovarian cancer, breast cancer, pancreatic cancer, prostate cancer, renal cell carcinoma, hepatic carcinoma, bile duct carcinoma, choriocarcinoma, seminoma, testicular tumor, Wilms' tumor, Ewing's tumor, bladder carcinoma, angiosarcoma, endotheliosarcoma, adenocarcinoma, sweat gland carcinoma, sebaceous gland sarcoma, papillary sarcoma, papillary adenosarcoma, cystadenosarcoma, bronchogenic carcinoma, medullar carcinoma, mastocytoma, mesotheliorma, synovioma, melanoma, leiomyosarcoma, rhabdomyosarcoma, neuroblastoma, retinoblastoma, oligodentroglioma, acoustic neuroma, hemangioblastoma, meningioma, pinealoma, ependymoma, craniopharyngioma, epithelial carcinoma, embryonic carcinoma, squamous cell carcinoma, base cell carcinoma, fibrosarcoma, myxoma, myxosarcoma, liposarcoma, chondrosarcoma, osteogenic sarcoma, leukemia, lymphoma, myeloma, and the metastatic lesions secondary to these primary tumors. A therapeutically effective dose refers to that amount of the compound sufficient to result in amelioration of symptoms of the disorders. Toxicity and therapeutic efficacy of such compounds can be determined by standard pharmaceutical procedures as described above.

A multidrug-resistant cell in which multidrug-resistance is inhibited can be further treated with a therapeutic agent to which the cell is no longer resistant or less resistant due to inhibition of the drug resistance activity.

Without further elaboration, it is believed that one skilled in the art can, based on the above disclosure and the examples below, utilize the present invention to its fullest extent. The following examples are to be construed as merely illustrative of how one skilled in the art can practice the invention, and are not limitative of the remainder of the disclosure in any way. All publications cited in this disclosure are hereby incorporated by reference.

### EXAMPLE 1

### Isolation of MRP3s1 cDNA

MRP3s1 polypeptide was identified while trying to characterize the cytoplasmic staining generated by monoclonal antibodies raised against a nucleolar protein, human checkpoint protein hRad17. See Bao S et al., Cancer Res., 59:2023, 1999. Whereas some of the antibodies showed expected nuclear staining, one of them, 25G10, showed a strong and peculiar cytoplasmic staining in colon and breast resistant cancer cell lines and tissues from refractory tumors.

In order to clone the gene encoding the protein recognized by 25G10, a HT-29 colon cancer cell expression library in the Uni-ZAP XR vector (# 937221, Stratagene, La Jolla, Calif.) was screened. About fifty thousand phage plaques were screened using 25G10 at a 1:100 dilution and anti-mouse HARP (Amersham Biosciences, Piscataway, N.J.) at 1:5000 dilution according to the method described in Ausubel, F. et al., eds. Current Protocols in Molecular Biology, John Wiley & Sons New York, 1999. Revelation was done using a Sigma Fast HRP detection kit (Sigma, St. Louis, Mo.). Six clones were purified, 5 of them represented the same gene based on restriction endonuclease digestion followed by agarose gel electrophoresis. The longest of these clones, 25G10-5 was sequenced on both strands using both plasmid and internal primers on an ABI PRISM® 377 DNA Sequencer (Applied Biosystems, Foster City, Calif.) at the Dana Farber Cancer Institute Sequencing Core Facility. This clone was found to contain an insert of 2242 base pairs (bp) nucleotides, which included an open reading frame (ORF) of 855 bp encoding a 285 amino acids putative protein.

### EXAMPLE 2

### Gene Analysis

Gene analysis revealed that the protein recognized by the 25G10 monoclonal antibody contained a putative ATP-binding cassette (ABC) ATPase domain, as well as one transmembrane domain. The protein was predicted to belong to the family of half-ABC transporters (Klein I. et al., Biochim Biophys Acta. 1461: 237-62, 1999). As hRad17 also possesses a related ATPase domain (know as “Walker-A” box), which is the only element that both hRad17 and this cloned novel half-ABC protein share, it is probable that the epitope recognized by 25G10 monoclonal antibody is centered around the ATPase domain.

Further analysis revealed that the 3′ sequence of the cDNA encoding the novel half-ABC protein was homologous to that of multidrug resistance protein 3, MRP3. This novel half-ABC protein was therefore annotated as MRP3 splice variant 1 (MRP3s1), as it shares the last 5 carboxyl-terminal exons of MRP3, but possesses a novel amino terminal exon of 750 bp.

The MRP3s1 gene was localized on chromosome 17q22 by BLAST searching of the human genome sequence using the “Human Genome Resources” tool at the National Center for Biotechnology Information.

### EXAMPLE 3

### Isolation of a Promoter Sequence of the MRP3s1 Gene

Analysis of the genomic sequence upstream of MRP3s1 first exon revealed a 276 bp region that showed significantly homology to sequences in the promoters of other multidrug resistance proteins. This highly conserved promoter domain in the genomic sequence upstream of MRP3s1 suggested that MRP3s1 was not a splice variant of MRP3, but was in fact a new separate gene transcribed from its own promoter.

To test this hypothesis, the sequence upstream of MRP3s1 was cloned by PCR amplification from human genomic DNA using primers 5′-ATGAAGGGCCTGGACCCCAG-3′ (SEQ ID NO:6) and 5′-TCCTTGCTAAGCTCCAAGAT-3′ (SEQ ID NO:7). Resultant PCR fragments were cloned in the PCR2.1 vector (Clontech, Palo Alto, Calif.) and their identity confirmed by DNA sequencing. One of these clones, MRPprom 5, contained the 276 bp sequence described above.

### EXAMPLE 4

### Cell-Type Specific Gene Expression Driven by the MRP3s1 Minimal Promoter

A 544 bp EcoRI fragment (SEQ ID NO:3) was isolated from MRPprom5 and cloned into the EcoRI site of the pd2EGFP vector (Clontech, Palo Alto, Calif.) in order to assess whether the resultant construct, MRP3s1-GFP, could direct the expression of the green fluorescent protein (GFP) once transfected in resistant cancer cells. MCF-7 breast cancer cells were seeded on glass coverslips and grown in 50:50 DMEM-RPMI 1640 medium (50% DMEM, 50% RPMI 1640) containing 10% fetal bovine serum (FBS) and 0.1% penicillin/streptomycin. The cells were transfected with 1-2 μg of DNA in 5 μl of Superfect reagent (Qiagen, Valencia, Calif.) according to the manufacturer's instructions. Following incubation at 37° C. for 2-3 hours, the DNA-Superfect complex was replaced with Dulbecco's modified Eagle's medium containing 10% FBS. 48 hours after transfection, the cells were fixed in 4% paraformaldehyde-phosphate-buffered saline (PBS) at room temperature for 30 minutes. The coverslips were then rinsed 3 times with PBS and mounted in anti-fade mounting media Crystal Mount, (Biomeda, Foster City, Calif.). The cells were then examined under an LSM410 confocal laser-scanning microscope (Carl Zeiss, Germany) at 488-nm argon excitation using a 515-nm-long pass barrier filter. Optical sections of 512×512 pixels were digitally recorded in the 2× line-averaging mode. Images were processed for reproduction using the Photoshop software (Adobe Systems, Mountainview, Calif.).

Green fluorescence could be observed in MRP3s1-GFP transfected cells whereas no fluorescence could be observed in the cells transfected with the pd2EGFP vector (without the MRP3s1 promoter). These results confirmed that MRP3s1 is indeed a new gene possessing its own promoter and regulatory elements.

### EXAMPLE 5

### Expression of the MRP3s1 Gene in Normal or Diseased Tissues or Cell Lines

The fact that MRP3s1 has its own promoter raises the possibility that this promoter might confer to MRP3s1 a unique pattern of expression in normal and diseased tissues. MRP3s1 expression was therefore evaluated by Northern blot analysis.

A riboprobe, containing the unique 5′ exon of the MRP3s1 gene, was prepared. In brief, a 553 bp PstI/BamHI restriction fragment of MRP3s1 was isolated from low melting agarose gel and 50 ng of the isolated DNA was used to generate a radioactive ³²P riboprobe using the T7 polymerase Rediprime™ DNA Labelling System (Amersham, Piscataway, N.J.) according to the manufacturer instructions. The radioactive probe thus produced was purified using G50 sepharose (Pharmacia) and hybridized to Multiple Tissue Northern Blots, MTN™ Cancer Panel (Clontech, Palo Alto, Calif.) according to the method described in Auclair D. et al., Am J. Physiol. 272 (3 Pt 1): C1007-16, 1997.

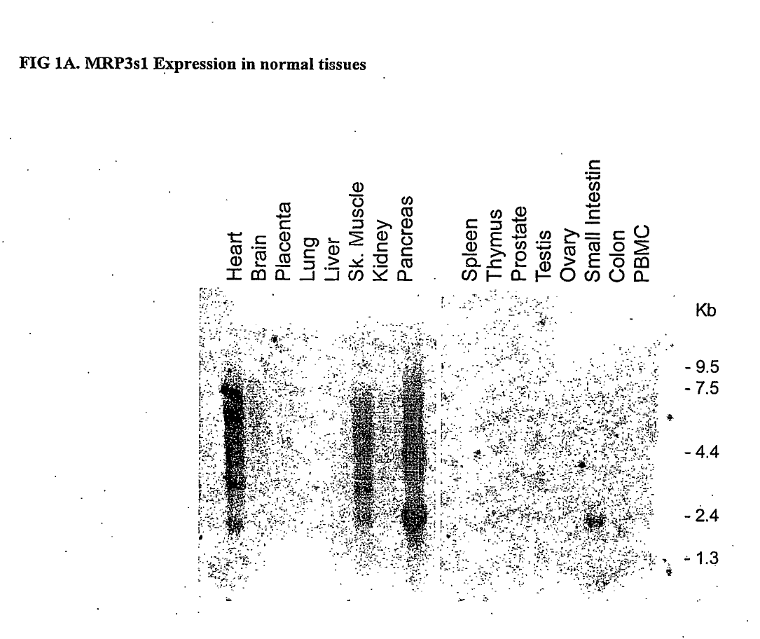

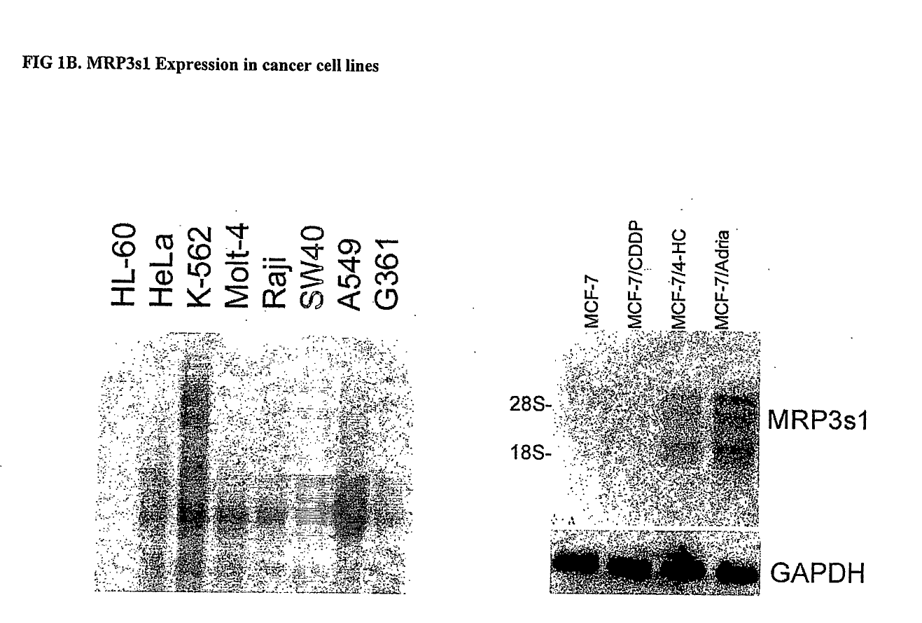

The results indicated that a 2.5 kb transcript of MRP3s1 was detected in pancreas and, to a less extend, in colon (FIG. 1A). It is very significant that MRP3s1 was expressed mostly in pancreas since carcinoma of the pancreas is the most refractory to any form of chemotherapy (Wagner M. et al., Ann Oncol. 10 Suppl 4:247-51, 1999). Similarly, colon cancer is also very refractory to chemotherapy, especially to DNA-damaging agents, such as alkylating agents and radiation (Gorlick R. et al., Semin Oncol. 26:606-11, 1999). MRP3s1 transcript was also found in a number of refractory cancer cell lines (FIG. 1B). Most cancer cell lines on the Multiple Tissue Northern Blot (MTN™) Cancer Panel (FIG. 1B, left panel) showed a 2.5 lb transcript that was most abundant in SW480 colon cancer and A549 lung carcinoma cell lines. Multidrug resistant variants of breast cancer cell line MCF-7 (Frei E. et al., Cancer Res 48: 6417-6423, 1988) were also tested by Northern (FIG. 1B, right panel). Whereas parental MCF-7 showed little expression of MRP3s1, multidrug resistant derivatives of MCF-7 showed a 2.5 kb MRP3s1 band as well as higher molecular weight bands.

### EXAMPLE 6

### Generation of Monoclonal Antibodies Against the MRP3s1 Polypeptide

The procedure for producing the hybridoma cell lines was essentially the same as that of Kohler and Milstein (Nature 256:495, 1975). Detailed procedure can be found in Harlow, E. et al., Antibodies: A Laboratory Manual, 1988, Cold Spring Harbor Laboratory Press, Cold Spring Harbor, N.Y.

Balb/c mice were immunized subcutaneously with the full length MRP3s1 polypeptide prepared in 0.4 ml complete (for the first injection on day 1) or incomplete (for boosting injection on day 18 and day 30) Freund's adjuvant. Two days after the last immunization, the mice were sacrificed and spleen cells were isolated. The spleen cells (2×10⁸) were fused with NS-1 mouse myeloma cells (2×10⁸) in the presence of 1 ml of 50% polyethylene glycol (PEG 1000, ATCC) in RPMI 1640 medium (GIBCO, Bethesda, Md.) and 1.5% DMSO for 2 minutes at 37° C. The cells were washed in RPMI 1640 medium, resuspended in 200 ml of complete medium (RPMI 1640 containing 15% fetal calf serum, 2 mM glutamine, 100 mg/ml garamycin), and plated in twenty 96-well plates (Flow Laboratories). The cells were fed with an HAT selection medium (13.6 mg/ml hypoxanthine, 0.18 mg/ml aminopterin, 3.9 mg/ml thymidine) for two weeks and then changed to an HAT selection medium (containing 13.6 mg/ml hypoxanthine and 3.9 mg/ml thymidine). Hybridoma cells thus produced were screened for anti-MRP3s1-1 antibodies using a standard method.

### EXAMPLE 7

### Detection of the MRP3s1 Polypeptide in Cancer Cells Using Western Blotting

Antibodies generated as described above were used to detect MRP3s1 polypeptide in HT-29 colon cancer cells by Western blotting.

HT-29 colon cancer cells were grown in 50:50 DMEM-RPMI medium (50% DMEM and 50% RPMI 1640) containing 10% FBS and 0.1% penicillin/streptomycin. After the cell cultures reached about 90% confluence, the cells were washes twice with PBS, scraped and solubilized in 0.5 ml of lysis buffer with protease inhibitors (50 mM Tris-HCl, pH 8.0, 150 mM NaCl, 0.5% Nonidet P-40, 10 mM NaF, 1 mM sodium orthovanadate, 1 mm dithiothreitol, 10 μg/ml leupeptin, 10 μg/ml aprotinin, 10 μg/ml trypsin/chymotrypsin inhibitor, 5 μg/ml pepstatin A, and 1 mM phenylmethylsulfonyl fluoride). The amount of polypeptide in each sample was quantified by a method based on Peterson's modification of the micro-Lowry method (Peterson, G. et al., Analyt. Biochem. 83, 346-356, 1977).

For Western blotting, 50 μg of proteins were loaded on a 7.5% SDS-PAGE and then electro-transferred to a Hybond-PVDF membrane at 4° C. The membrane was blocked overnight at room temperature in a TNT buffer (prepared by dissolving 8.8 g of NaCl, 10 ml of Tris 1 N pH 8.0, and 500 μl Tween 20 in 1 liter of ultrapure water) containing 5% non-fat dry milk. The membrane was then rinsed 3 times for 10 minutes in a TNT buffer containing 0.1% bovine serum albumin (BSA), and then incubated with a supernatant of hybridoma clone diluted at 1:100 in TNT-3% BSA at room temperature for 1 hour. After washing in TNT-0.1% BSA, the membrane was incubated with a secondary sheep anti-mouse antibody conjugated to horseradish peroxidase (1:5,000 dilution, Amersham Pharmacia Biotech, Piscataway, N.J.). After washing in TNT-0.1% BSA, the blots were visualized by chemiluminescence using the ECL kit from Amersham Pharmacia Biotech.

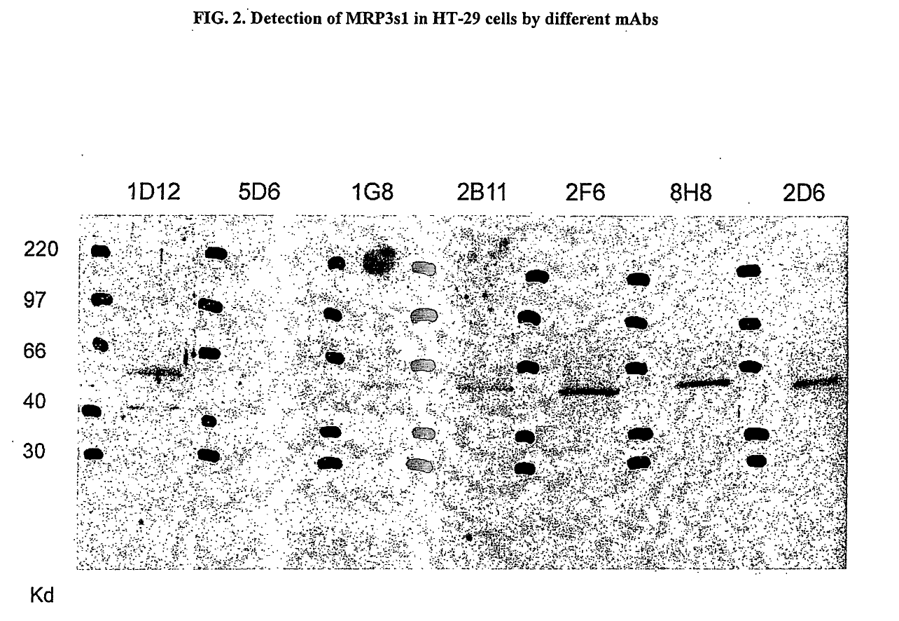

As shown in FIG. 2, all hybridoma supernatants recognized a major protein band of approximately 70 kDa. The supernatant of hybridoma clone 1E12 also recognized a lower band of approximately 40 kDa. Since half-ABC proteins are usually present in cells as tight homo- or heterodimers (Klein I. et al., Biochim Biophys Acta. 1461:237-62, 1999), it is probable that the 70 kDa band was the dimeric form of MRP3s1, whereas the 40 kDa was the monomeric form.

### EXAMPLE 8

### Immunofluorescence

HT29 cells were grown on coverslips 50:50 medium (50% DMEM, 50% RPMI 1640 containing 10% FBS and 0.1% penicillin/streptomycin). The cells were fixed in methanol at −20° C. for 2 minutes and permeabilized with 0.1% Triton X-100 prepared-PBS for 10 minutes. Alternatively, the cells were fixed with 2% paraformaldehyde-PBS at 20° C. for 10 minutes and permeabilized with 0.1% triton X-100 for 10 minutes. After the cells were then blocked for 30 minutes in a blocking solution containing 1% BSA, and 1% mouse serum (Sigma, St. Louis, Mo.) in PBS, the cells were incubated sequentially for 60 minutes each with 1:10 diluted hybridoma supernatant and 1:100 diluted rhodamine-coupled anti-mouse IgG antibodies (Jackson ImmunoResearch Laboratories, West Grove, Pa.). Between each step, the coverslips were washed in PBS-0.3% BSA for 5 minutes.

The stained cells were then examined under an LSM410 confocal laser scanning microscope (Carl Zeiss, Germany) equipped with an argon-krypton laser (568 nm). Optical sections of 512×512 pixels were digitally recorded in the 2× line-averaging mode. Images were processed for reproduction using the Photoshop software (Adobe Systems, Mountainview, Calif.). The results are summarized in Table 1.

TABLE 1
Results of immunofluorescence staining

| | methanol fixation | paraformaldehyde fixation |
|---|---|---|
| 1H7 | — | — |
| 2B11 | — | — |
| 6D4 | — | — |
| 9H6 | — | — |
| 1A10 | — | — |
| 1G8 | — | — |
| 3D5 | — | — |
| 7D12 | — | — |
| 9D12 | — | — |
| 1E12 | label throughout cell, | weak nuclear label, dotted |
| | possibly surface | pattern, nucleoli excluded |
| 5E3 | — | — |
| 8B7 | — | — |
| 2B5 | — | — |
| 2F6 | — | — |
| 5D6 | cytoplasmic network labeled, | cytoplasmic network labeled, |
| | cytoskeleton? | cytoskeleton? |
| 8H9 | — | — |
| 2D6 | (small nuclear dots, very weak) | (very weak staining, surface?) |
| PBS | — | — |
| control | | |

Note:
“—” means negative staining.

As shown in Table 1, cells stained with three hybridomas, 1E12, 5D6, and 2D6, showed positive staining. More specifically, 1E12 labeled throughout the cell, and possibly the cell surface when methanol was used to fix cells. When paraformaldehyde was used to fix cells, 1E12 staining showed a weak, and probably unspecific, dotted pattern in nucleus (except in nucleoli). 5D6 labeled throughout the cell cytoplasm, especially, along cytoskeleton structures, when cells were fixed with methanol or paraformaldehyde. This pattern is similar to that revealed by 25G10 monoclonal antibody described in Example 1 above. 2D6 revealed a very weak undefined cytoplasmic staining upon methanol fixation and a weak cell surface staining upon paraformaldehyde fixation.

### EXAMPLE 9

### Drug-Resistance Rendered by the Expression of MRP3s1

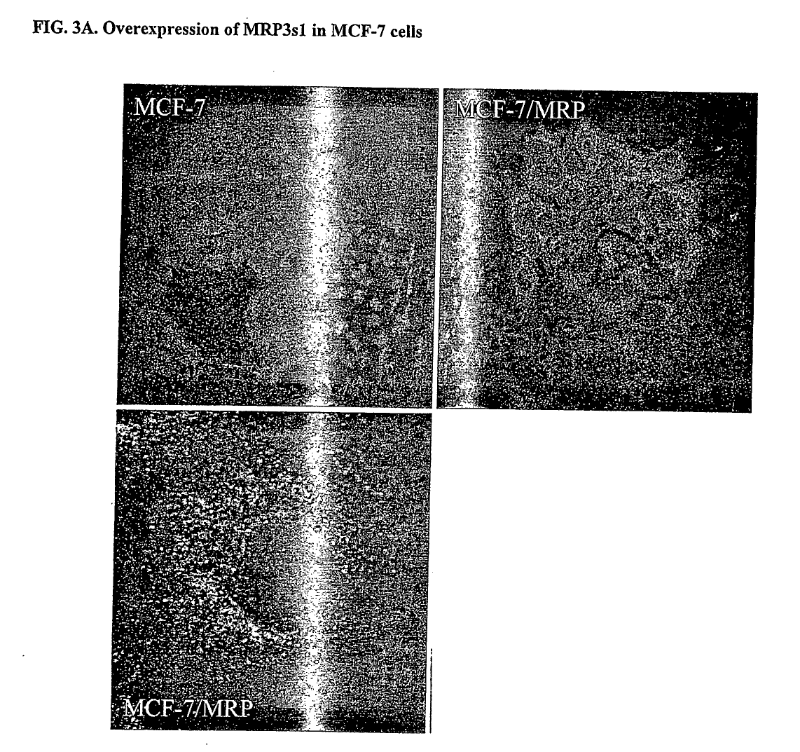

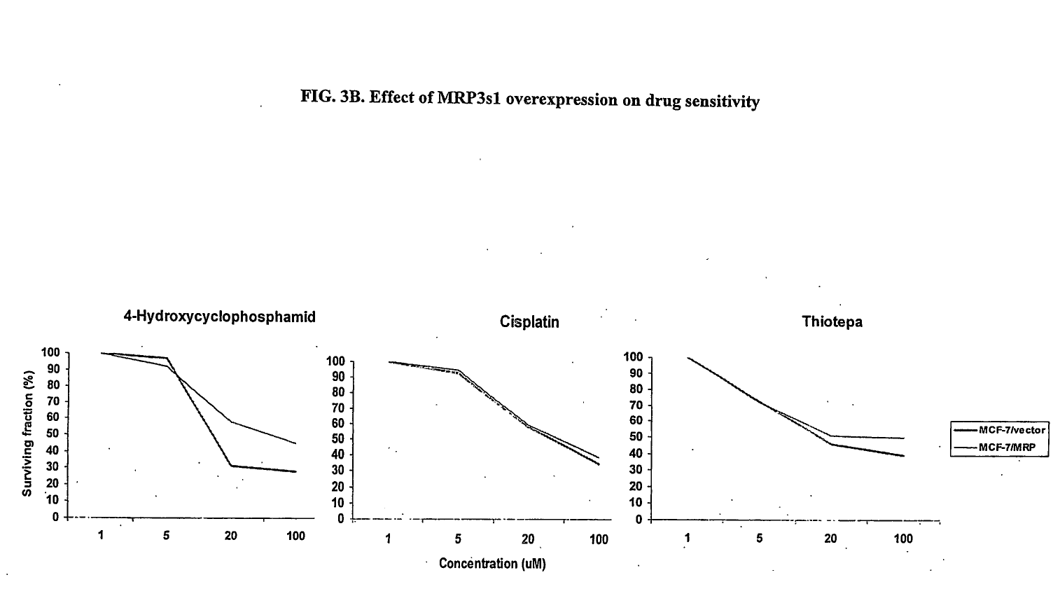

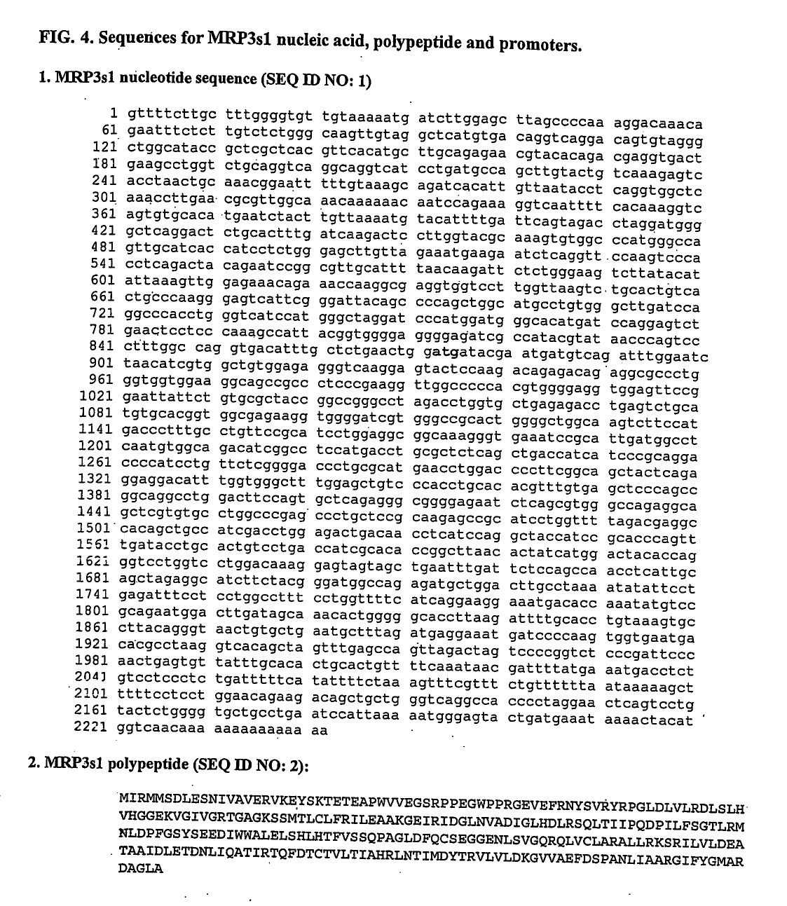

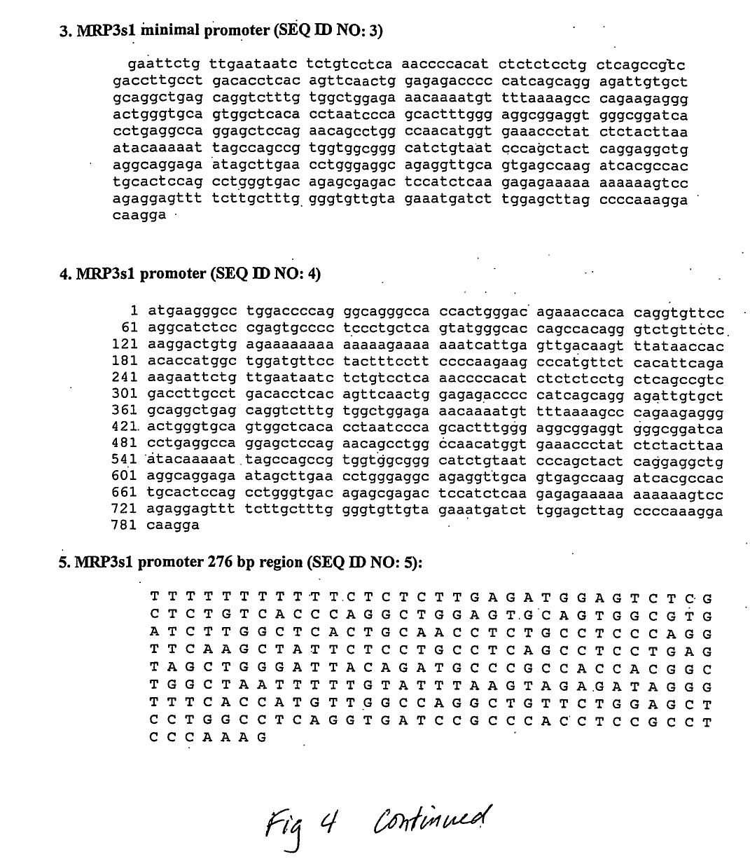

To investigate whether MRP3s1 could render cells resistant to DNA-damaging agents, MCF-7 cells were stably transfected with an MRP3s1 expression vector, MRP3s1/Bsd. To generate this vector, an MRP3s1 full length EcoRI-XhoI fragment was cloned into the EcoRI-XhoI sites of the pcDNA6/Bsd vector (Clontech, Palo Alto, Calif.). MCF-7cells were transfected with the MRP3s1/Bsd vector and an empty pcDNA/Bsd vector, respectively, by the method described in Example 4 above. Stable transfectants were selected through serial passages under blasticidin (Clontech, Palo Alto, Calif.) selection at 10 μg/ml]. Cell clones were isolated and repicked under the same selection pressure. One of the clones expressed the highest amount of MRP3s1 by immunofluorescence staining (FIG. 3A). This clone was further tested by the MTS/MTT assays described in Cory A. et al., Cancer Commun. 7:207-12, 1991 and found to be 3-4 times more resistant to DNA damaging agents, such as 4-hydroxycyclophosphamide, than MCF-7 cells stably transfected with the empty pcDNA/Bsd control vector (FIG. 3B).

It is to be understood that while the invention has been described in conjunction with the detailed description thereof, the foregoing description is intended to illustrate and not limit the scope of the invention, which is defined by the appended claims. Other aspects, advantages, and modifications are within the scope of this invention.

What is claimed is:

1. An isolated nucleic acid comprising a nucleotide sequence which is at least 70% identical to the sequence of SEQ ID NO:3.

2. An isolated nucleic acid comprising a nucleotide sequence which is at least 80% identical to the sequence of SEQ ID NO:3.

3. An isolated nucleic acid comprising a nucleotide sequence Which is at least 90% identical to the sequence of SEQ ID NO:3.

4. An isolated nucleic acid comprising a nucleotide sequence which is at least 95% identical to the sequence of SEQ ID NO:3.

5. An isolated nucleic acid comprising the nucleotide sequence of SEQ ID NO: 3.

6. The nucleic acid of claim 5 further comprising a nucleotide sequence encoding a polypeptide with the amino acid sequence of SEQ ID NO:2.

7. An expression vector comprising the nucleic acid of claim 6 operably linked to an expression control sequence.

8. A cultured cell comprising the vector of claim 7.

9. A method for producing a polypeptide, comprising culturing the cultured cell of claim 8 in a medium under conditions permitting expression of a polypeptide encoded by the vector, and purifying the polypeptide from the cultured cell or the medium of the cell.

10. A cultured cell transfected with the expression vector of claim 7, or a progeny of the cultured cell, wherein the cultured cell expresses a polypeptide encoded by the expression vector.

11. A cultured cell comprising the nucleic acid of claim 6 operably linked to an expression control sequence.

12. A method for producing a polypeptide, comprising culturing the cultured cell of claim 11 in a medium under conditions permitting expression under the control of the expression control sequence, and purifying a polypeptide encoded by the nucleic acid from the cell or the medium of the cell.

13. The nucleic acid of claim 5 further comprising SEQ ID NO:1 or fragments thereof.

14. An isolated nucleic acid comprising a sequence that hybridizes under low stringency conditions to a hybridization probe the sequence of which consists of SEQ ID NO:1 or the complement of SEQ ID NO:1.

15. An isolated nucleic acid comprising a sequence that hybridizes under medium stringency conditions to a hybridization probe the sequence of which consists of SEQ ID NO:1 or the complement of SEQ ID NO:1.

16. An isolated nucleic acid comprising a sequence that hybridizes under high stringency conditions to a hybridization probe the sequence of which consists of SEQ ID NO:1 or the complement of SEQ ID NO:1.

17. An isolated polypeptide comprising an amino acid sequence which is at least 70% identical to the amino acid sequence of SEQ ID NO:2.

18. The isolated polypeptide of claim 17, wherein the polypeptide, when expressed in a cell, renders the cell resistant to DNA-damaging agents.

19. An expression vector, comprising:
a first nucleic acid sequence comprising SEQ ID NO:3; and
a second nucleic acid sequence encoding a gene,
wherein the 5′-end of the second sequence is operatively linked to the 3′-end of the first sequence.

20. The vector of claim 19, wherein the gene encodes a green fluorescent protein, a luciferase, or a lacZ.

21. The vector of claim 19, wherein the gene encodes a suicide protein.

22. A cultured cell comprising the vector of claim 19.

23. A purified antibody that binds specifically to a polypeptide with the amino acid sequence of SEQ ID NO:2 or fragments thereof.

24. A nucleic acid sequence comprising SEQ ID NO: 3 operably linked to a heterologous sequence.

25. A method for detecting a cellular proliferative disorder in a subject, comprising:
i) providing a test sample of a subject; and
ii) measuring the expression level of a gene encoding a polypeptide with a sequence of SEQ ID NO:2 (MRP3s1 gene) in the test sample.

26. The method of claim 25 further comprising reporting the expression level of the MRP3s1 gene in the test sample.

27. The method of claim 26 further comprising comparing the expression level to a predetermined value.

28. The method of claim 25, wherein the expression level of the MRP3s1 gene is the amount of an mRNA encoding a polypeptide with a sequence of SEQ ID NO:2.

29. The method of claim 25, wherein the expression level of the MRP3s1 gene is the amount of a polypeptide with a sequence of SEQ ID NO:2.

30. The method of claim 28 further comprising
i) contacting an antibody against a polypeptide that comprises a sequence of SEQ ID NO:2 with a cell in the test sample; and
ii) detecting binding of the antibody.

31. A method for monitoring a subject undergoing a therapeutic treatment, comprising:
i) obtaining a test sample from a subject; and
ii) measuring the expression level of a gene encoding a polypeptide with a sequence of SEQ ID NO:2 (MRP3s1 gene) in the test sample.

32. The method of claim 31 further comprising obtaining a previous sample from a subject at an earlier time.

33. The method of claim 32 further comprising reporting the expression levels in the test sample and the previous sample.

34. A method for targeting a cellular proliferative disorder in a subject, comprising:
i) identifying a subject suffering a cellular proliferative disorder; and
ii) administering to the subject an agent that can bind to a polypeptide comprising the amino acid sequence of SEQ ID NO:2 or fragments thereof.

35. A method for expressing a foreign polypeptide in a cell in vivo, wherein the foreign polypeptide can bind to a polypeptide with the amino acid sequence of SEQ ID NO:2, comprising
i) providing an expression vector encoding the foreign polypeptide;
ii) introducing the vector into a cell in vivo; and
iii) maintaining the cell in vivo under conditions permitting expression of the foreign polypeptide in the cell.

36. A method for introducing a foreign nucleic acid into a cell in vivo, comprising:
i) providing a sequence comprising the foreign nucleic acid; and
ii) contacting the sequence with a cell in vivo,
wherein the foreign nucleic acid is complementary to SEQ ID NO:1 or fragments thereof

37. A method for targeting a cellular proliferative disorder in a subject, comprising
i) identifying a subject having a cellular proliferative disorder; and
ii) administering to the subject an agent that can bind to a nucleic acid comprising the nucleotide sequence of SEQ ID NO: 3.

38. A method for targeting a cellular proliferative disorder in a subject, comprising
i) identifying a subject having a cellular proliferative disorder; and
ii) administering to the subject an agent that can modulate the expression level of a gene encoding to a polypeptide comprising the amino acid sequence of SEQ ID NO:2.

39. A method for modulating the cellular pump mechanism of a resistant tumor cell, comprising
i) providing an agent that binds to a polypeptide comprising the amino acid sequence of SEQ ID NO:2, or fragments thereof; and
ii) contacting the agent with the cell.

40. A method for modulating the cellular pump mechanism of a resistant tumor cell in a subject, comprising administering to a subject having a resistant tumor cell an agent that binds to a polypeptide comprising the amino acid sequence of SEQ ID NO: 2.

41. A method for screening for a therapeutic agent for treating a drug-resistant tumor cell, comprising:
i) providing a cell system comprising a reporter gene operatively linked to a regulatory sequence constructed and arranged to drive the transcription of the reporter gene;
ii) contacting the cell system with a candidate agent; and
iii) measuring the level of synthesis of the gene product of the reporter gene, wherein a decreased level of synthesis in the presence of the candidate agent compared to in the absence of the agent is indicative of the agent being an effective agent for treating a drug-resistant tumor cell.

42. The method of claim 41, wherein the regulatory sequence comprises SEQ ID NO:3.

43. The method of claim 41, wherein the reporter gene encodes a green fluorescent protein, a luciferase, or a lacZ.

44. A cell for screening for a therapeutic agent for treating a drug-resistant tumor cell, wherein the cell comprises a reporter gene operatively linked to a sequence constructed and arranged to drive the transcription of the reporter gene.

45. The cell of claim 44, wherein the sequence comprises SEQ ID NO:3.

46. The cell of claim 44, wherein the reporter gene encodes a green fluorescent protein, a luciferase, or a lacZ.

47. A method for making an antibody, comprising immunizing a non-human animal with an immunogenic fragment of a polypeptide with the sequence of SEQ ID NO:2.

48. A method for making an antibody, comprising providing a hybridoma cell that produces a monoclonal antibody specific for a polypeptide with the sequence of SEQ ID NO: 2, and culturing the cell under conditions that permit production of the monoclonal antibody.

49. A method for modulating expression of a gene responsible for controlling cellular pump mechanisms in cell, comprising
i) providing an effective amount agent that binds to a nucleic acid comprising the nucleotide sequence of SEQ ID NO: 3 or fragments thereof; and
ii) contacting the agent with the cell.

50. A method for delivering a suicide protein to a tumor cell, comprising
i) providing an expression vector comprising a first nucleic acid with the sequence of SEQ ID NO:3 and a second nucleic acid sequence encoding a suicide protein, wherein the second sequence is operatively linked to the first sequence; and
ii) contacting the vector with the cell.


SEQUENCE LISTING (7 sequences)

SEQ ID NO: 1 - DNA, 2242 bp, Homo sapiens
gttttcttgctttggggtgttgtaaaaatgatcttggagcttagccccaaaggacaaacagaatttctcttgtctctgggcaagttgtaggctcatgtgacaggtcaggacagtgtagggctggcataccgctcgctcacgttcacatgcttgcagagaacgtacacagacgaggtgactgaagcctggtctgcaggtcaggcaggtcatcctgatgccagcttgtactgtcaaagagtcacctaactgcaaacggaatttttgtaaagcagatcacattgttaatacctcaggtggctcaaaccttgaacgcgttggcaaacaaaaaacaatccagaaaggtcaattttcacaaaggtcagtgtgcacatgaatctacttgttaaaatgtacattttgattcagtagacctaggatggggctcaggactctgcactttgatcaagactccttggtacgcaaagtgtggcccatgggccagttgcatcaccatcctctgggagcttgttagaaatgaagaatctcaggttccaagtcccacctcagactacagaatccggcgttgcattttaacaagattctctgggaagtcttatacatattaaagttggagaaacagaaaccaaggcgaggtggtccttggttaagtctgcactgtcactgcccaagggagtcattcgggattacagccccagctggcatgcctgtgggcttgatccaggcccacctgggtcatccatgggctaggatcccatggatgggcacatgatccaggagtctgaactcctcccaaagccattacggtggggaggggagatcgccatacgtataacccagtccctttggccaggtgacatttgctctgaactggatgatacgaatgatgtcagatttggaatctaacatcgtggctgtggagagggtcaaggagtactccaagacagagacagaggcgccctgggtggtggaaggcagccgccctcccgaaggttggcccccacgtggggaggtggagttccggaattattctgtgcgctaccggccgggcctagacctggtgctgagagacctgagtctgcatgtgcacggtggcgagaaggtggggatcgtgggccgcactggggctggcaagtcttccatgaccctttgcctgttccgcatcctggaggcggcaaagggtgaaatccgcattgatggcctcaatgtggcagacatcggcctccatgacctgcgctctcagctgaccatcatcccgcaggaccccatcctgttctcggggaccctgcgcatgaacctggaccccttcggcagctactcagaggaggacatttggtgggctttggagctgtcccacctgcacacgtttgtgagctcccagccggcaggcctggacttccagtgctcagagggcggggagaatctcagcgtgggccagaggcagctcgtgtgcctggcccgagccctgctccgcaagagccgcatcctggttttagacgaggccacagctgccatcgacctggagactgacaacctcatccaggctaccatccgcacccagtttgatacctgcactgtcctgaccatcgcacaccggcttaacactatcatggactacaccagggtcctggtcctggacaaaggagtagtagctgaatttgattctccagccaacctcattgcagctagaggcatcttctacgggatggccagagatgctggacttgcctaaaatatattcctgagatttcctcctggcctttcctggttttcatcaggaaggaaatgacaccaaatatgtccgcagaatggacttgatagcaaacactgggggcaccttaagattttgcacctgtaaagtgccttacagggtaactgtgctgaatgctttagatgaggaaatgatccccaagtggtgaatgacacgcctaaggtcacagctagtttgagccagttagactagtccccggtctcccgattcccaactgagtgttatttgcacactgcactgttttcaaataacgattttatgaaatgacctctgtcctccctctgatttttcatattttctaaagtttcgtttctgttttttaataaaaagctttttcctcctggaacagaagacagctgctgggtcaggccacccctaggaactcagtcctgtactctggggtgctgcctgaatccattaaaaatgggagtactgatgaaataaaactacatggtcaacaaaaaaaaaaaaaaa

SEQ ID NO: 2 - protein, 285 aa, Homo sapiens
MIRMMSDLESNIVAVERVKEYSKTETEAPWVVEGSRPPEGWPPRGEVEFRNYSVRYRPGLDLVLRDLSLHVHGGEKVGIVGRTGAGKSSMTLCLFRILEAAKGEIRIDGLNVADIGLHDLRSQLTIIPQDPILFSGTLRMNLDPFGSYSEEDIWWALELSHLHTFVSSQPAGLDFQCSEGGENLSVGQRQLVCLARALLRKSRILVLDEATAAIDLETDNLIQATIRTQFDTCTVLTIAHRLNTIMDYTRVLVLDKGVVAEFDSPANLIAARGIFYGMARDAGLA

SEQ ID NO: 3 - DNA, 544 bp, Homo sapiens
gaattctgttgaataatctctgtcctcaaaccccacatctctctcctgctcagccgtcgaccttgcctgacacctcacagttcaactggagagacccccatcagcaggagattgtgctgcaggctgagcaggtctttgtggctggagaaacaaaatgttttaaaagcccagaagagggactgggtgcagtggctcacacctaatcccagcactttgggaggcggaggtgggcggatcacctgaggccaggagctccagaacagcctggccaacatggtgaaaccctatctctacttaaatacaaaaattagccagccgtggtggcgggcatctgtaatcccagctactcaggaggctgaggcaggagaatagcttgaacctgggaggcagaggttgcagtgagccaagatcacgccactgcactccagcctgggtgacagagcgagactccatctcaagagagaaaaaaaaaaagtccagaggagttttcttgctttggggtgttgtagaaatgatcttggagcttagccccaaaggacaagga

SEQ ID NO: 4 - DNA, 786 bp, Homo sapiens
atgaagggcctggaccccagggcagggccaccactgggacagaaaccacacaggtgttccaggcatctcccgagtgcccctccctgctcagtatgggcaccagccacagggtctgttctcaaggactgtgagaaaaaaaaaaaaagaaaaaaatcattgagttgacaagtttataaccacacaccatggctggatgttcctactttccttccccaagaagcccatgttctcacattcagaaagaattctgttgaataatctctgtcctcaaaccccacatctctctcctgctcagccgtcgaccttgcctgacacctcacagttcaactggagagacccccatcagcaggagattgtgctgcaggctgagcaggtctttgtggctggagaaacaaaatgttttaaaagcccagaagagggactgggtgcagtggctcacacctaatcccagcactttgggaggcggaggtgggcggatcacctgaggccaggagctccagaacagcctggccaacatggtgaaaccctatctctacttaaatacaaaaattagccagccgtggtggcgggcatctgtaatcccagctactcaggaggctgaggcaggagaatagcttgaacctgggaggcagaggttgcagtgagccaagatcacgccactgcactccagcctgggtgacagagcgagactccatctcaagagagaaaaaaaaaaagtccagaggagttttcttgctttggggtgttgtagaaatgatcttggagcttagccccaaaggacaagga

SEQ ID NO: 5 - DNA, 263 bp, Homo sapiens
tttttttttttctctcttgagatggagtctcgctctgtcacccaggctggagtgcagtggcgtgatcttggctcactgcaacctctgcctcccaggttcaagctattctcctgcctcagcctcctgagtagctgggattacagatgcccgccaccacggctggctaatttttgtatttaagtagagatagggtttcaccatgttggccaggctgttctggagctcctggcctcaggtgatccgcccacctccgcctcccaaag

SEQ ID NO: 6 - DNA, 20 bp, Artificial Sequence
  Primer
atgaagggcctggaccccag

SEQ ID NO: 7 - DNA, 20 bp, Artificial Sequence
  Primer
tccttgctaagctccaagat